SEQUENCE LISTING (14 sequences)

SEQ ID NO: 1 - protein, 9 aa, Artificial Sequence
  synthetic peptide
VMAPRTLLL

SEQ ID NO: 2 - protein, 15 aa, Artificial Sequence
  synthetic peptide
ARAASLSLGFLFLLF

SEQ ID NO: 3 - protein, 15 aa, Artificial Sequence
  synthetic peptide
KELKFVTLVFRHGDR

SEQ ID NO: 4 - protein, 15 aa, Artificial Sequence
  synthetic peptide
QLTQLGMEQHYELGE

SEQ ID NO: 5 - protein, 15 aa, Artificial Sequence
  synthetic peptide
LNESYKHEQVYIRST

SEQ ID NO: 6 - protein, 15 aa, Artificial Sequence
  synthetic peptide
NHMKRATQMPSYKKL

SEQ ID NO: 7 - protein, 15 aa, Artificial Sequence
  synthetic peptide
MVLLFIHIRRGPCWQ

SEQ ID NO: 8 - protein, 15 aa, Artificial Sequence
  synthetic peptide
VPEPASQHTLRSGPG

SEQ ID NO: 9 - protein, 15 aa, Artificial Sequence
  synthetic peptide
SAERLQGRRSRGASG

SEQ ID NO: 10 - protein, 15 aa, Artificial Sequence
  synthetic peptide
IDESLIFYKKWELEA

SEQ ID NO: 11 - protein, 15 aa, Artificial Sequence
  synthetic peptide
PFTYEQLDVLKHKLD

SEQ ID NO: 12 - protein, 15 aa, Artificial Sequence
  synthetic peptide
FMKLRTDAVLPLTVA

SEQ ID NO: 13 - protein, 15 aa, Artificial Sequence
  synthetic peptide
LQGRRSRGASGSEPQ

SEQ ID NO: 14 - protein, 15 aa, Artificial Sequence
  synthetic peptide
HEDPMGQQGSLGEQQ

(19) United States
(12) Patent Application Publication
(10) Pub. No.: US 2022/0257740 A1
(43) Pub. Date: Aug. 18, 2022

(54) TUMOR-ASSOCIATED ANTIGEN-SPECIFIC T CELL RESPONSES

(71) Applicant: Oregon Health & Science University, Portland, OR (US)

(72) Inventors: Klaus J. FRÜH, Portland, OR (US); Scott G. HANSEN, Portland, OR (US); Louis J. PICKER, Portland, OR (US)

(21) Appl. No.: 17/616,941

(22) PCT Filed: Jun. 5, 2020
(86) PCT No.: PCT/US2020/036481

§ 371 Date: Dec. 6, 2021

Related U.S. Application Data
(60) Provisional application No. 62/858,756, filed on Jun. 7, 2019.
(60) Provisional application No. 62/878,511, filed on Jul. 25, 2019.

(51) Int. Cl. A61K 39/00; A61K 9/00; A61P 35/00

(52) U.S. Cl. CPC A61K 39/001153; A61K 39/001193; A61K 9/0019; A61K 2039/605; A61K 2039/5158; A61K 2039/53; A61P 35/00

(57) ABSTRACT

The present disclosure relates to antigens and methods of generating an immune response for the treatment of cancer. The disclosure also relates to methods of generating MHC-Ia, MHC-II, and/or MHC-E restricted CD8+ T cells for the treatment or prevention of cancer.

### CROSS REFERENCE TO RELATED APPLICATIONS

This application claims the benefit of U.S. Provisional Application No. 62/878,511, filed Jul. 25, 2019, which claims the benefit of U.S. Provisional Application No. 62/858,756, filed Jun. 7, 2019, each of which are hereby incorporated by reference in their entirety.

### STATEMENT REGARDING FEDERALLY-SPONSORED RESEARCH AND DEVELOPMENT

This invention was made with government support under grant numbers R44 CA180177-03 awarded by the National Cancer Institute. The government has certain rights in the invention.

### REFERENCE TO SEQUENCE LISTING SUBMITTED ELECTRONICALLY

The content of the electronically submitted sequence listing in ASCII text file (Name 4153_012PC02_SL_ST25; Size: 3,591 bytes; and Date of Creation: May 27, 2020) filed with the application is incorporated herein by reference in its entirety.

### BACKGROUND OF THE INVENTION

Cytomegalovirus (CMV)-based vaccines, as well as other herpesvirus-based vaccines, are on the horizon as a promising addition to our arsenal against infectious disease and cancer. These herpesvirus-based vectors are unique, not only in the high level of T cell immunity they induce against their heterologous encoded pathogen (or cancer) target antigen, but also in the durability of the immunity and in its “immediate-effector” quality.

Many tumor-associated antigens (TAA) are self-antigens that are abnormally expressed by cancer cells. The main challenge for eliciting TAA-specific T cell responses is that, as self-antigens, “canonical” T cells that strongly recognize peptides derived from these antigens in the context of MHC-I or MHC-II are removed from the immune repertoire by negative selection. Thus, an effective cancer vaccine should break immunological tolerance by stimulating “non-canonical” T cells that have escaped negative selection by either expressing low affinity TCRs or by recognizing peptides binding with low affinity to the MHC. All currently available T cell inducing vaccines, e.g., DNA, RNA, poxvector, adenovectors, or alphavirus-based, are designed to elicit canonical T cells. As a result, there remains a need in the art for therapeutic approaches capable of breaking immunological tolerance to tumor-associated antigens.

### BRIEF SUMMARY OF THE INVENTION

The present disclosure relates to methods of generating an immune response to a tumor-associated antigen in a subject, the method comprising administering to the subject a CMV vector encoding a tumor-associated antigen in an amount effective to elicit a CD8+ T cell response to the tumor-associated antigen, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The present disclosure also relates to methods of treating cancer in a subject, the method comprising administering to the subject a CMV vector encoding a tumor-associated antigen in an amount effective to elicit a CD8+ T cell response to the tumor-associated antigen, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The present disclosure also relates to CMV vectors encoding a tumor-associated antigen for use in generating an immune response to the tumor-associated antigen in a subject, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The present disclosure also relates to CMV vectors encoding a tumor-associated antigen for use in the treatment of cancer in a subject, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The present disclosure also relates to use of CMV vectors encoding a tumor-associated antigen in the manufacture of a medicament for use in generating an immune response to the tumor-associated antigen in a subject, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The present disclosure also relates to use of CMV vectors encoding a tumor-associated antigen in the manufacture of a medicament for the treatment of cancer, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The present disclosure also relates to methods of treating cancer caused by a tumor virus in a subject, comprising administering to the subject a CMV vector encoding a tumor virus antigen in an amount effective to elicit a CD8+ T cell response to the tumor-associated antigen, wherein the CMV vector does not express an active UL128, UL130, UL146, and UL147 protein or orthologs thereof.

The present disclosure also relates to methods of treating cancer caused by a tumor virus in a subject, the method comprising administering to the subject a CMV vector encoding a tumor virus antigen in an amount effective to elicit a CD8+ T cell response to the tumor virus antigen, wherein the CMV vector does not express an active UL128, UL130, UL146, and UL147 protein or orthologs thereof.

The present disclosure also relates to CMV vectors encoding a tumor virus antigen for use in the treatment of cancer in a subject, wherein the CMV vector does not express an active UL128, UL130, UL146, and UL147 protein or orthologs thereof.

The present disclosure also relates to use of a CMV vectors encoding a tumor virus antigen in the manufacture of a medicament for the treatment of cancer, wherein the CMV vector does not express an active UL128, UL130, UL146, and UL147 protein or orthologs thereof.

In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14). In some embodiments, the tumor-associated antigen comprises the amino acid sequence ARAASLSLGFLFLLF (SEQ ID NO: 2). In some embodiment, the tumor-associated antigen comprises the amino acid sequence KELKFVTLVFRHGDR (SEQ ID NO: 3). In some embodiments, the tumor-associated antigen comprises the amino acid sequence QLTQLGMEQHYELGE (SEQ ID NO: 4). In some embodiments, the tumor-associated antigen comprises the amino acid sequence LNESYKHEQVYIRST (SEQ ID NO: 5). In some embodiments, the tumor-associated antigen comprises the amino acid sequence NHMKRATQMPSYKKL (SEQ ID NO: 6). In some embodiments, the tumor-associated antigen comprises the amino acid sequence MVLLFIHIRRGPCWQ (SEQ ID NO: 7). In some embodiments, the tumor-associated antigen comprises the amino acid sequence VPEPASQHTLRSGPG (SEQ ID NO: 8). In some embodiments, the tumor-associated antigen comprises the amino acid sequence SAERLQGRRSRGASG (SEQ ID NO: 9). In some embodiments, the tumor-associated antigen comprises the amino acid sequence IDESLIFYKKWELEA (SEQ ID NO: 10). In some embodiments, the tumor-associated antigen comprises the amino acid sequence PFTYEQLDVLKHKLD (SEQ ID NO: 11). In some embodiments, the tumor-associated antigen comprises the amino acid sequence FMKLRTDAVLPLTVA (SEQ ID NO: 12). In some embodiments, the tumor-associated antigen comprises the amino acid sequence LQGRRSRGASGSEPQ (SEQ ID NO: 13). In some embodiments, the tumor-associated antigen comprises the amino acid sequence HEDPMGQQGSLGEQQ (SEQ ID NO: 14).

In some embodiments, at least 10% of the CD8+ T cells elicited by the CMV vector are restricted by MHC-E or an ortholog thereof, or MHC-II or an ortholog thereof. In another embodiment, at least 20%, at least 30%, at least 40%, at least 50%, at least 60%, or at least 75% of the CD8+ T cells elicited by the CMV vector are restricted by MHC-E or an ortholog thereof. In another embodiment, fewer than 10% of the CD8+ T cells elicited by the CMV vector are restricted by MHC-class 1a or an ortholog thereof. In another embodiment, some of the CD8+ T cells restricted by MHC-E recognize peptides that are shared by at least 90% of other subjects immunized with the vector.

In some embodiments, the specific MHC-E supertopes comprise peptides derived from prostatic acidic phosphatase epitopes. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 5. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO:6. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 8. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 9. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 13. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 14.

In some embodiments, some of the CD8+ T cells restricted by MHC-II recognize peptides shared by at least 90% of other subjects immunized with the vector.

In some embodiments, the peptides comprise peptides derived from prostatic acidic phosphatase epitopes. In another embodiment, the MHC-II epitope has at least 50%, at least 60%, at least 70%, at least 75%, at least 80%, at least 85%, at least 90%, or at least 95% identity to the amino acid sequence corresponding to SEQ ID NO: 2. In another embodiment, the MHC-II epitope has at least 50%, at least 60%, at least 70%, at least 75%, at least 80%, at least 85%, at least 90%, or at least 95% identity to the amino acid sequence corresponding to SEQ ID NO: 3. In another embodiment, the MHC-II epitope has at least 50%, at least 60%, at least 70%, at least 75%, at least 80%, at least 85%, at least 90%, or at least 95% identity to the amino acid sequence corresponding to SEQ ID NO: 4. In another embodiment, the MHC-II epitope has at least 50%, at least 60%, at least 70%, at least 75%, at least 80%, at least 85%, at least 90%, or at least 95% identity to the amino acid sequence corresponding to SEQ ID NO: 7.

The present disclosure also relates to methods of generating CD8+ T cells that recognize MHC-E-tumor-associated antigen peptide complexes, the method comprising: (a) administering to a first subject a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-E/tumor-associated antigen-derived peptide complex; (c) isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-E/tumor-associated antigen peptide complexes.

The present disclosure also relates to methods of generating CD8+ T cells that recognize MHC-E-tumor-associated antigen peptide complexes, the method comprising: (a) isolating from a first subject a first set of CD8+ T cells, wherein the first subject has been administered a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-E/tumor-associated antigen-derived peptide complex; (c) isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-E/tumor-associated antigen peptide complexes.

In some embodiments, the recombinant CMV vector is a recombinant human CMV vector or a recombinant rhesus macaque CMV vector.

In some embodiments, the tumor-associated antigen is related to a cancer selected from the group consisting of prostate cancer, kidney cancer, mesothelioma, breast cancer, and cervical cancer. In another embodiment, the tumor-associated antigen is selected from the group consisting of prostatic acidic phosphatase, Wilms tumor suppressor protein, mesothelin, and Her-2, or orthologs thereof.

In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14). In some embodiments, the tumor-associated antigen comprises the amino acid sequence ARAASLSLGFLFLLF (SEQ ID NO: 2). In some embodiment, the tumor-associated antigen comprises the amino acid sequence KELKFVTLVFRHGDR (SEQ ID NO: 3). In some embodiments, the tumor-associated antigen comprises the amino acid sequence QLTQLGMEQHYELGE (SEQ ID NO: 4). In some embodiments, the tumor-associated antigen comprises the amino acid sequence LNESYKHEQVYIRST (SEQ ID NO: 5). In some embodiments, the tumor-associated antigen comprises the amino acid sequence NHMKRATQMPSYKKL (SEQ ID NO: 6). In some embodiments, the tumor-associated antigen comprises the amino acid sequence MVLLFIHIRRGPCWQ (SEQ ID NO: 7). In some embodiments, the tumor-associated antigen comprises the amino acid sequence VPEPASQHTLRSGPG (SEQ ID NO: 8). In some embodiments, the tumor-associated antigen comprises the amino acid sequence SAERLQGRRSRGASG (SEQ ID NO: 9). In some embodiments, the tumor-associated antigen comprises the amino acid sequence IDESLIFYKKWELEA (SEQ ID NO: 10). In some embodiments, the tumor-associated antigen comprises the amino acid sequence PFTYEQLDVLKHKLD (SEQ ID NO: 11). In some embodiments, the tumor-associated antigen comprises the amino acid sequence FMKLRTDAVLPLTVA (SEQ ID NO: 12). In some embodiments, the tumor-associated antigen comprises the amino acid sequence LQGRRSRGASGSEPQ (SEQ ID NO: 13). In some embodiments, the tumor-associated antigen comprises the amino acid sequence HEDPMGQQGSLGEQQ (SEQ ID NO: 14).

In some embodiments, the first CD8+ T cell recognizes specific MHC-E supertopes. In another embodiment, the specific MHC-E supertopes comprise peptides derived from prostatic acidic phosphatase epitopes. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 5. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 6. In some embodiments, the specific MHC-E supertopes comprise peptides derived from Wilms tumor suppressor protein epitopes. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 8. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 9. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 13. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 14.

In some embodiments, the second CD8+ T cell recognizes specific MHC-E supertopes. In another embodiment, the specific MHC-E supertopes comprise peptides derived from prostatic acidic phosphatase epitopes. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 5. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 6. In another embodiment, the specific MHC-E supertopes comprise peptides derived from Wilms tumor suppressor protein epitopes. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 8. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 9. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 13. In another embodiment, the MHC-E supertope has at least 50%, at least 60%, at least 70%, at least 75%, at least 80%, at least 85%, at least 90%, or at least 95% identity to the amino acid sequence corresponding to SEQ ID NO: 14.

In some embodiments, the first CD8+ TCR is identified by DNA or RNA sequencing.

In some embodiments, the nucleic acid sequence encoding the second CD8+ TCR is identical to the nucleic acid sequence encoding the first CD8+ TCR.

In some embodiments, the first subject is a human or nonhuman primate. In another embodiment, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In another embodiment, the second CD8+ TCR comprises the non-human primate CDR1α, CDR2α, CDR3α, CDR1β, CDR2β, and CDR3β of the first CD8+ TCR. In another embodiment, the second CD8+ TCR comprises CDR1α, CDR2α, CDR3α, CDR1β, CDR2β, and CDR3β of the first CD8+ TCR. In another embodiment, the nucleic acid sequence encoding the second CD8+ TCR is identical to the nucleic acid sequence encoding the first CD8+ TCR.

In some embodiments, the second CD8+ TCR is a chimeric CD8+ TCR. In another embodiment, the second CD8+ TCR comprises CDR1α, CDR2α, CDR3α, CDR1β, CDR2β, and CDR3β of the first CD8+ TCR.

In some embodiments, administering the CMV vector to the first subject comprises intravenous, intramuscular, intraperitoneal, or oral administration of the CMV vector to the first subject. In another embodiment, the transfected CD8+ T cells are administered to the second subject to treat or prevent cancer. In another embodiment, the cancer is prostate cancer, kidney cancer, mesothelioma, breast cancer, or cervical cancer.

The present disclosure also relates to methods of CD8+ T cells that recognize MHC-II-tumor peptide complexes, the method comprising: (a) administering to a first subject a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-II/tumor antigen-derived peptide complex; (c)isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-II/tumor antigen peptide complexes.

The present disclosure also relates to methods of generating CD8+ T cells that recognize MHC-II-tumor antigen peptide complexes, the method comprising: (a)

isolating from a first subject a first set of CD8+ T cells, wherein the first subject has been administered a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof, (b)

identifying a first CD8+ TCR from the first set of CD8+ T cells, wherein the first CD8+ TCR recognizes a MHC-II/tumor antigen-derived peptide complex; (c)

isolating a second set of one or more CD8+ T cells from a second subject; and (d)

transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-II/tumor antigen peptide complexes.

In some embodiments, the at least one recombinant CMV vector is a recombinant human CMV vector or a recombinant rhesus macaque CMV vector.

In some embodiments, the at least one recombinant CMV vector does not express an active UL128 protein, or ortholog thereof, does not express an active UL130 protein, or ortholog thereof, does not express and active UL146, or ortholog thereof, does not express an active UL147, or ortholog thereof, and does not express an active US11 protein, or ortholog thereof. In another embodiment, the mutations in the nucleic acid sequence encoding UL128, UL130, UL146, UL147, or US11 are selected from the group consisting of point mutations, frameshift mutations, truncation mutations, and deletion of all of the nucleic acid sequence encoding the viral protein.

In some embodiments, the tumor-associated antigen is related to a prostate cancer, kidney cancer, mesothelioma, breast cancer, or cervical cancer. In another embodiment, the tumor-associated antigen is prostatic acidic phosphatase, Wilms tumor suppressor protein, mesothelin, or Her-2, or orthologs thereof.

In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14). In some embodiments, the tumor-associated antigen comprises the amino acid sequence ARAASLSLGFLFLLF (SEQ ID NO: 2). In some embodiment, the tumor-associated antigen comprises the amino acid sequence KELKFVTLVFRHGDR (SEQ ID NO: 3). In some embodiments, the tumor-associated antigen comprises the amino acid sequence QLTQLGMEQHYELGE (SEQ ID NO: 4). In some embodiments, the tumor-associated antigen comprises the amino acid sequence LNESYKHEQVYIRST (SEQ ID NO: 5). In some embodiments, the tumor-associated antigen comprises the amino acid sequence NHMKRATQMPSYKKL (SEQ ID NO: 6). In some embodiments, the tumor-associated antigen comprises the amino acid sequence MVLLFIHIRRGPCWQ (SEQ ID NO: 7). In some embodiments, the tumor-associated antigen comprises the amino acid sequence VPEPASQHTLRSGPG (SEQ ID NO: 8). In some embodiments, the tumor-associated antigen comprises the amino acid sequence SAERLQGRRSRGASG (SEQ ID NO: 9). In some embodiments, the tumor-associated antigen comprises the amino acid sequence IDESLIFYKKWELEA (SEQ ID NO: 10). In some embodiments, the tumor-associated antigen comprises the amino acid sequence PFTYEQLDVLKHKLD (SEQ ID NO: 11). In some embodiments, the tumor-associated antigen comprises the amino acid sequence FMKLRTDAVLPLTVA (SEQ ID NO: 12). In some embodiments, the tumor-associated antigen comprises the amino acid sequence LQGRRSRGASGSEPQ (SEQ ID NO: 13). In some embodiments, the tumor-associated antigen comprises the amino acid sequence HEDPMGQQGSLGEQQ (SEQ ID NO: 14).

In some embodiments, the first CD8+ T cell recognizes a MHC-II supertope.

In some embodiments, the MHC-II supertope comprises a peptide derived from a prostatic acidic phosphatase epitope. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 2. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 3. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 4. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 7.

In some embodiments, the second CD8+ T cell recognizes a MHC-II supertope.

In some embodiments, the MHC-II supertope comprises a peptide derived from a prostatic acidic phosphatase epitope. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 2. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 3. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 4. In another embodiment, the MHC-II supertope has at least 50%, at least 60%, at least 70%, at least 75%, at least 80%, at least 85%, at least 90%, or at least 95% identity to the amino acid sequence corresponding to SEQ ID NO: 7.

In some embodiments, the first CD8+ TCR is identified by DNA or RNA sequencing.

In some embodiments, the nucleic acid sequence encoding the second CD8+ TCR is identical to the nucleic acid sequence encoding the first CD8+ TCR.

In some embodiments, the first subject is a human or nonhuman primate. In another embodiment, the second subject is a human or nonhuman primate.

In some embodiments, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In another embodiment, the second CD8+ TCR comprises the non-human primate CDR1α, CDR2α, CDR3α, CDR1β, CDR2β, and CDR3β of the first CD8+ TCR. In another embodiment, the second CD8+ TCR comprises CDR1α, CDR2α, CDR3α, CDR1β, CDR2β, and CDR3β of the first CD8+ TCR. In another embodiment, the nucleic acid sequence encoding the second CD8+ TCR is identical to the nucleic acid sequence encoding the first CD8+ TCR. In another embodiment, the second CD8+ TCR is a chimeric CD8+ TCR. In another embodiment, the second CD8+ TCR comprises CDR1α, CDR2α, CDR3α, CDR1β, CDR2β, and CDR3β of the first CD8+ TCR.

In some embodiments, administering the CMV vector to the first subject comprises intravenous, intramuscular, intraperitoneal, or oral administration of the CMV vector to the first subject. In another embodiment, the transfected CD8+ T cells are administered to the second subject to treat cancer. In another embodiment, the cancer is a prostate cancer, kidney cancer, mesothelioma, breast cancer, or cervical cancer.

In some embodiments, a CD8+ T cell is generated. In another embodiment, the CD8+ T cell is administered to the subject to treat or prevent cancer. In another embodiment, the CD8+ T cell is administered to the subject to induce an immune response to a host self-antigen. In some embodiments, the CD8+ T cell is used in the manufacture of a medicament for the treatment or prevention of cancer. In some embodiments, the CD8+ T cell is administered to a subject to induce an immune response to a host self-antigen. In some embodiments, the CD8+ T cell is used in inducing an immune response to a host self-antigen in a subject. In some embodiments, the CD8+ T cell is used in the manufacture of a medicament for inducing an immune response to a host self-antigen.

The present disclosure also relates to an isolated MHC-E or MHC-II supertope peptide between about 8 and about 15 amino acids in length that is capable of being recognized by CD8+ T cell receptors, wherein the supertope comprises a tumor-associated antigen.

In some embodiments, the peptide is ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12) LQGRRSRGASGSEPQ (SEQ ID NO: 13); or HEDPMGQQGSLGEQQ (SEQ ID NO: 13).

The present disclosure also relates to methods of overcoming immune tolerance to a tumor-associated antigen in a subject in need thereof, comprising administering an effective amount of a cytomegalovirus (CMV) vector that expresses the tumor-associated antigen to the subject.

In some embodiments, the CMV vector is a human CMV vector or a rhesus macaque CMV vector.

In some embodiments, the CMV vector does not express active UL128, or orthologs thereof, does not express active UL130, or orthologs thereof, does not express active UL146, or orthologs thereof, and does not express active UL147, or orthologs thereof. In another embodiment, the CMV vector does not express active UL128, UL130, UL146, or UL147, or orthologs thereof, due to the presence of one or more mutations in the nucleic acid sequence encoding UL238, UL130, UL146, or UL147. In another embodiment, the mutations in the nucleic acid sequence encoding UL128, UL130, UL146, or UL147 are one or more of point mutations, frameshift mutations, truncation mutations, and deletion of all of the nucleic acid sequence encoding the viral protein.

In some embodiments, the CMV vector is rhesus macaque CMV strain 68-1.

In some embodiments, the CMV vector does not express an active UL82 protein, or ortholog thereof. In another embodiment, the CMV vector does not express an active UL82 protein, or ortholog thereof, due to the presence of one or more mutations in the nucleic acid sequence encoding UL82. In another embodiment, the mutations in the nucleic acid sequence encoding UL82 are one or more of point mutations, frameshift mutations, truncation mutations, and deletion of all of the nucleic acid sequence encoding UL82.

In some embodiments, the tumor-associated antigen is derived from a prostate cancer, kidney cancer, mesothelioma, breast cancer, or cervical cancer. In another embodiment, the tumor-associated antigen is prostatic acidic phosphatase, Wilms tumor suppressor protein, mesothelin, or Her-2.

In some embodiments, the effective amount comprises an amount effective to elicit a CD8+ T cell response to the tumor-associated antigen in the subject.

In some embodiments, at least 10%, at least 20%, at least 30%, at least 40%, or at least 50% of the CD8+ T cells are restricted by MHC-I or an ortholog thereof.

In some embodiments, a CD8+ TCR is identified from the CD8+ T cells elicited by the CMV vector, wherein the CD8+ TCR recognizes a MHC-I/tumor-associated antigen-derived peptide complex. In another embodiment, wherein the CD8+ TCR is identified by DNA or RNA sequencing. In some embodiments, the subject is a human.

### BRIEF DESCRIPTION OF THE DRAWINGS/FIGURES

FIG. 1 shows the average T cell response frequencies elicited in six rhesus macaques (RM) inoculated with RhCMV strain 68-1 expressing the cancer antigen PAP (68-1/PAP) and the SIV gag antigen (68-1/SIVgag). Three of the RM were additionally co-inoculated with a RhCMV strain 68-1 expressing the cancer antigen WT1 (68-1/WT1) and the other three were co-inoculated with a RhCMV strain 68-1 expressing the cancer antigen MSLN (68-1/MSLN). CD4+ and CD8+ T cell responses were measured in peripheral blood mononuclear cells (PBMC) using overlapping peptide pools for each of the antigens by intracellular cytokine staining (ICS) at each of the indicated time points. The average response frequencies are shown.

FIG. 2 shows the average T cell response frequencies elicited in six female RM inoculated with RhCMV strain 68-1 expressing the cancer antigen HER2 (68-1/HER2). CD4+ and CD8+ T cell responses were measured in peripheral blood mononuclear cells using overlapping peptide pools for HER2 by intracellular cytokine staining at each of the indicated time points. The average response frequencies are shown.

FIG. 3 shows the average T cell response frequencies elicited in eight female RM inoculated with either RhCMV strain 68-1 (68-1/HPV) (solid lines) or RhCMV strain 68-1.2 (68-1.2/HPV) (dashed lines) expressing a fusion protein of the E6 and E7 proteins of HPV16 and HPV18. CD4+ and CD8+ T cell responses were measured in PBMC using overlapping peptide pools for HPV antigens by ICS at each of the indicated time points. The individual response frequencies are shown.

FIG. 4 shows the MHC-E-dependent recognition of HPV antigens by CD8+ T cells from RM immunized with 68-1/HPV. Four female RM were inoculated with 68-1 expressing a fusion protein of the E6 and E7 proteins of HPV16 and HPV18 (see FIG. 3). T cell responses were measured by ICS for TNFα and IFNγ. CD8+ T cells responding with both TNFα and IFNγ production appear in the upper right quadrant. VMAPRTLLL (SEQ ID NO: 1) (VL9) is a MHC-E ligand peptide.

FIG. 5 shows the MHC-E-dependent recognition of PAP by CD8+ T cells from RM immunized with 68-1/PAP. CD8+ T cells were isolated and co-incubated with K562 cells expressing MHC-E and K562 cells expressing MHC-E and PAP or MHC-E and the HPV fusion protein. T cell responses were measured by ICS for TNFα and IFNγ. CD8+ T cells responding with both TNFα and IFNγ production appear in the upper right quadrant. VMAPRTLLL (SEQ ID NO: 1) (VL9) is a MHC-E ligand peptide.

FIG. 6 shows MHC-E-dependent recognition of PAP and WT1 by CD8+ T cells from RM immunized with 68-1/PAP and 68-1/WT1. Six male RM were co-inoculated with 68-1/PAP and 68-1/WT1. CD8+ T cells were isolated and co-incubated with K562 cells expressing MHC-E and K562 cells expressing MHC-E and PAP or MHC-E and WT1. T cell responses were measured by ICS for TNFα and IFNγ.

FIG. 7 shows the CD4+ and CD8+ T cell responses in PBMC were measured by ICS using overlapping peptide pools for TNFα and IFNγ at the indicated time points. The frequency of PAP-specific T cells among memory T cells is shown.

FIG. 8 shows the MHC-restriction analysis of PAP-specific CD8+ T cells. CD8+ T cell responses to individual peptides are shown as squares along the PAP sequences. Peptide responses blocked by MHC-I specific antibody W6/32 indicate MHC-I restriction, peptide responses blocked by MHC-II specific peptide CLIP indicate MHC-II restriction, and peptide responses blocked by MHC-E-specific peptide indicate MHC-E restriction. Peptides that have not yet been tested in the presence of blocking reagents are also indicated. Gray box: restriction pending; white box: conventional MHC-Ia restricted; dotted box: unconventional MHC-E restricted; dashed box: unconventional MHC-II restricted; black box: blocking indeterminate.

FIG. 9 shows the peptide mapping of PAP, WT1, and MSLN-specific CD8+ T cells. CD8+ T cell responses to individual peptides are shown as squares along the sequences. Peptides that have not yet been tested in the presence of blocking reagents. “Supertope”-peptides that resulted in responses in all 68-1/PAP animals are boxed: grey dashed box: MHC-II or MHC-E supertope, black box: MHC-II supertopes (based on results from FIG. 8), black dashed box: MHC-E supertopes (based on results from FIG. 8).

FIG. 10 shows the MHC-restriction analysis of WT1-specific CD8+ T cells. CD8+ T cell responses to individual peptides are shown as square long the WT1 sequence. Peptide responses blocked by MHC-I specific antibody W6/32, peptide responses blocked by MHC-II specific peptide CLIP, peptide responses blocked by MHC-E-specific peptide, peptides that have not yet been tested in the presence of blocking reagents. The following “Supertope”-peptides resulted in responses in all 68-1/WT1-immunized animals: #3, #13, #14, #58. Results obtained in three animals suggest that all of these supertopes are MHC-E restricted. Gray box: restriction pending; black box: conventional MHC-IA restricted; white box: unconventional MHC-IE restricted; dashed box: unconventional MHC-II restricted; dotted box: blocking indeterminate.

### DETAILED DESCRIPTION OF THE INVENTION

### I. Terms

Unless otherwise noted, technical terms are used according to conventional usage.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

Although methods and materials similar or equivalent to those described herein may be used in the practice or testing of this disclosure, suitable methods and materials are described below. In addition, the materials, methods, and examples are illustrative only and not intended to be limiting. In order to facilitate review of the various embodiments of the disclosure, the following explanations of specific terms are provided.

Unless the context requires otherwise, throughout the present specification and claims, the word “comprise” and variations thereof, such as “comprises” and “comprising,” are to be construed in an open, inclusive sense, that is, as “including, but not limited to”. “Consisting of” shall mean excluding more than trace elements of other ingredients and substantial method steps disclosed herein. The term “consisting essentially of” limits the scope of a claim to the specified materials or steps, or to those that do not materially affect the basic characteristics of a claimed invention. For example, a composition consisting essentially of the elements as defined herein would not exclude trace contaminants from the isolation and purification method and pharmaceutically acceptable carriers, such as phosphate buffered saline, preservatives, and the like. Similarly, a protein consists essentially of a particular amino acid sequence when the protein includes additional amino acids that contribute to at most 20% of the length of the protein and do not substantially affect the activity of the protein (e.g., alters the activity of the protein by no more than 50%). Embodiments defined by each of the transitional terms are within the scope of this invention.

About: As used herein, the term “about” may mean within 1%, 5%, 10% or 20% of a defined value.

Antigen: As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

Antigen-specific T cell: A CD8⁺ or CD4⁺ lymphocyte that recognizes a particular antigen. Generally, antigen-specific T cells specifically bind to a particular antigen presented by MHC molecules, but not other antigens presented by the same MHC.

Administration: As used herein, the term “administration” means to provide or give a subject an agent, such as a composition comprising an effective amount of a CMV vector comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal, and inhalation routes.

Effective amount: As used herein, the term “effective amount” refers to an amount of an agent, such as a CMV vector comprising a heterologous antigen or a transfected CD8+ T cell that recognizes a MHC-E/heterologous antigen-derived peptide complex, a MHC-II/heterologous antigen-derived peptide complex, or a MHC-I/heterologous antigen-derived peptide complex, that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

Heterologous antigen: As used herein, the term “heterologous antigen” refers to any protein or fragment thereof that is not derived from CMV. Heterologous antigens may be pathogen-specific antigens, tumor virus antigens, tumor-associated antigens, host self-antigens, or any other antigen.

Hyperproliferative disease: A disease or disorder characterized by the uncontrolled proliferation of cells. Hyperproliferative diseases include, but are not limited to malignant and non-malignant tumors.

Immune tolerance: As used herein “immune tolerance” refers to a state of unresponsiveness of the immune system to substances that have the potential to induce an immune response. Self-tolerance to an individual's own antigens, for example, tumor-associated antigens, is achieved through both central tolerance and peripheral tolerance mechanisms.

Epitope: As used herein “epitope” comprises an allele-specific motif or other sequence, such as an N-terminal repeat, such that a peptide comprising the motif will bind an MHC molecule and induce a cytotoxic T lymphocyte (“CTL”) response, or a B cell response (for example antibody production) against the antigen from which the epitope is derived.

In some embodiments, epitopes are identified using sequence motifs or other methods, such as neural net or polynomial determinations known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an epitope, a “conserved residue” is one which appears in a significantly higher frequency than would be expected by random distribution at a particular position in a peptide. In some embodiments, a conserved residue is one where the MHC structure may provide a contact point with the epitope.

Mutation: As used herein, the term “mutation” refers to any difference in a nucleic acid or polypeptide sequence from a normal, consensus, or “wild type” sequence. A mutant is any protein or nucleic acid sequence comprising a mutation. In addition, a cell or an organism with a mutation may also be referred to as a mutant. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence.

Nucleotide sequences or nucleic acid sequences: The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex.

Operably Linked: As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

Promoter: As used herein, the term “promoter” may refer to any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector.

Recombinant: As used herein, the term “recombinant” with reference to a nucleic acid or polypeptide refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

Operably linked: As used herein, the term “operably linked” means a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. For the disclosed tumor-associated antigens to be expressed, the protein coding sequence of the tumor-associated antigen should be “operably linked” to regulatory or nucleic acid control sequences that direct transcription and translation of the protein.

Pharmaceutically acceptable carriers: As used herein, a “pharmaceutically acceptable carrier” of use is conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Polynucleotide: As used herein, the term “polynucleotide” refers to a polymer of ribonucleic acid (RNA) or deoxyribonucleic acid (DNA). A polynucleotide is made up of four bases; adenine, cytosine, guanine, and thymine/uracil (uracil is used in RNA). A coding sequence from a nucleic acid is indicative of the sequence of the protein encoded by the nucleic acid.

Polypeptide: The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

Orthologs of proteins are typically characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

Promoter: As used herein, the term “promoter” refers to a group of transcriptional control modules that are clustered around the initiation site for RNA polymerase II and that when operationally linked to the protein coding sequences of the disclosure lead to the expression of the encoded protein. The expression of the transgenes of the present disclosure may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue or organ. Many suitable promoters and enhancers are known in the art, and any such suitable promoter or enhancer may be used for expression of the transgenes of the disclosure. For example, suitable promoters and/or enhancers may be selected from the Eukaryotic Promoter Database (EPDB).

Sequence identity/similarity: As used herein, the identity/similarity between two or more nucleic acid sequences, or two or more amino acid sequences, is expressed in terms of the identity or similarity between the sequences. Sequence identity may be measured in terms of percentage identity; the higher the percentage, the more identical the sequences are. Sequence similarity may be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) may be called “homologs.”

Sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer App Biosci 8, 155-165 (1992); and Pearson et al, Meth Mol Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) (Altschul et al, (1990) supra) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information may be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment may be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and may possess sequence identities of at least 85%, 90%, 95%, or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence may be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

Subject: As used herein, the term “subject” refers to a living multi-cellular vertebrate organisms, a category that includes both human and non-human mammals. The term “subject” includes all animals, including non-human primates and humans, while “animal” includes all vertebrate species, except humans; and “vertebrate” includes all vertebrates, including animals (as “animal” is used herein) and humans. And, of course, a subset of “animal” is “mammal”, which for purposes of this specification includes all mammals, except humans.

Supertope: As used herein, the term “supertope” or “supertope peptide” refers to an epitope or peptide that is recognized by T cells in greater than about 90% of the human population regardless of MHC haplotype, i.e., in the presence or absence of given MHC-I, MHC-II, or MHC-E alleles.

Tumor-associated antigen: As used herein, the term “tumor-associated antigen” refers to a self-antigen that is abnormally expressed by cancer cells. TAAs include: (i) germline/testis antigens expressed in cancer cells, (ii) cell lineage differentiation antigens not expressed in adult tissue, or (iii) antigens overexpressed in cancer cells. Tumor-associated antigens are relatively restricted to tumor cells and can be any protein that induces an immune response. However, many tumor-associated antigens are host (self) proteins and thus are typically not seen as antigenic by the host immune system. Tumor-associated antigens can also be abnormally expressed by cancer cells. Tumor-associated antigens can also be germline/testis antigens expressed in cancer cells, cell lineage differentiation antigens not expressed in adult tissue, or antigens overexpressed in cancer cells

Tumor virus: As used herein, the terms “tumor virus”, “cancer virus”, or “oncovirus” refers to a virus that, in some cases, induces development of cancer (e.g., after a chronic infection, in individuals with a compromised immune system, or the like).

Treatment: As used herein, the term “treatment” refers to an intervention that ameliorates a sign or symptom of a disease or pathological condition. As used herein, the terms “treatment,” “treat,” and “treating,” with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

Vaccine: An immunogenic composition that can be administered to a mammal, such as a human, to confer immunity, such as active immunity, to a disease or other pathological condition. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine is a replication-deficient CMV expressing a heterologous antigen, such as a tumor-associated antigen derived from a tumor of the lung, prostate, ovary, breast, colon, cervix, liver, kidney, bone, or a melanoma.

Vector: Nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

Any vector that allows expression of the viruses of the present disclosure may be used in accordance with the present disclosure. In certain embodiments, the disclosed viruses may be used in vitro (such as using cell-free expression systems) and/or in cultured cells grown in vitro in order to produce the encoded heterologous antigen (e.g., tumor virus antigens, HIV antigens, tumor-associated antigens, and antibodies) which may then be used for various applications such as in the production of proteinaceous vaccines. For such applications, any vector that allows expression of the virus in vitro and/or in cultured cells may be used. The vectors used in accordance with the present disclosure may contain a suitable gene regulatory region, such as a promoter or enhancer, such that the antigens of the disclosure may be expressed.

### II. Methods for the Treatment and Prevention of Cancer

Disclosed herein are methods for the treatment or prevention of cancer. The methods involve administering an effective amount of at least one recombinant CMV vector comprising at least one tumor associated antigen or tumor virus antigen to a subject. In some embodiments, the methods also comprise administration of a T cell comprising a MHC-E restricted T cell receptor.

Animal experiments have demonstrated that CMV vaccines are unique in that they: a) induce and maintain high frequencies of extralymphoid T cell responses (so called effector memory T cells); b) super-infect CMV-positive hosts; and c) maintain immunogenicity even when rendered deficient in host-to-host spread. Furthermore, experiments in animal models have shown that vaccine vectors derived from animal CMVs induce a protective immune response against infectious diseases and cancer (US 20080199493; US 20100142823; US 20130136768; and US 20140141038). Particularly striking is the finding that a rhesus CMV (RhCMV)-vectored simian immunodeficiency virus (SIV)-vaccine was able to not only prevent AIDS in non-human primates, but ultimately cure these animals from SIV (Hansen S G et al., Nature 502, 100-104 (2013)).

The main challenges for eliciting tumor-associated antigen (TAA)-specific T cells is that, as self-antigens, “canonical” T cells that strongly recognize peptides derived from these antigens in the context of MHC-I or MHC-II, have been removed from the immune repertoire by negative selection. Thus, a cancer vaccine must break immunological tolerance by stimulating “non-canonical” T cells that have escaped negative selection by either expressing low affinity TCRs or by recognizing peptides binding with low affinity to the MHC. All currently available T cell inducing vaccines, e.g., DNA, RNA, poxvector, adenovectors, or alphavirus-based, are designed to elicit canonical T cells. As a result, these vectors have difficulties with breaking immunological tolerance. Moreover, anti-vector immunity prevents the repeated use of the same vector to boost immunity resulting in complicated heterologous prime/boost vaccine regimens or they need to be combined with tolerance-breaking checkpoint inhibitors. For instance, PROSTVAC is a poxvirus-based vaccine against prostate cancer expressing PSA (prostate specific antigen). The immunization regimen requires one immunization with PSA expressed by vaccinia virus followed by six booster immunizations with fowlpoxvirus encoding PSA. Despite this effort, PROSTVAC was only able to elicit CD8+ T cells representing about 0.03% of total CD8+ T cells. As a result, a phase III clinical trial was stopped due to futility.

In some embodiments, the method provides treating a cancer related to a tumor-associated antigen. In some embodiments, the treatment results from breaking tolerance in the subject such that an immune response is raised against the TAA.

In some embodiments, the cancer is caused by a pathogen. In some embodiments, the pathogen is a tumor virus and the antigen is a protein derived from the tumor virus. Tumor viruses include, but are not limited to, human T-lymphotropic virus, hepatitis B virus, hepatitis C virus, human papillomavirus (HPV), human polyomavirus, Kaposi's sarcoma-associated herpesvirus, merkel cell polyomavirus, and Epstein-barr virus. In some embodiments the tumor virus antigen is E6 and E7 from HPV strain 16 or E6 and E7 from HPV strain 18. In some embodiments the tumor virus antigen is a fusion of E6 and E7 from HPV. The tumor virus antigen may be a protein derived from any portion of the tumor virus. For example, in some embodiments, the tumor virus antigen may be derived from the core, envelope, surface, or polymerase proteins.

Tumor-associated antigens include, but are not limited to, prostatic acidic phosphatase (PAP); Wilms tumor suppressor protein (WT1); Mesothelin (MSLN); Her-2 (HER2); human papilloma virus antigen E6 of strain HPV16; human papilloma virus antigen E7 of strain HPV16; human papilloma virus antigen E6 of strain HPV18; Human papilloma virus antigen E7 of strain HPV18; a fusion protein of human papilloma virus E6 and E7 from HPV16 and HPV18; mucin 1 (MUC1); LMP2; epidermal growth factor receptor (EGFR); p53; New York esophagus 1 (NY-ESO-1); prostate specific membrane antigen (PSMA); GD2, carcinoembryonic antigen (CEA); melanoma antigen a/melanoma antigen recognized by T cells 1 (MelanA/MART1); Ras; gp100, Proteinase 3 (PR1), Bcr-abl; Survivin; prostate specific antigen (PSA); human telomerase reverse transcriptase (hTERT); EphA2; ML-IAP; alpha-fetoprotein (AFP); EpCAM; ERG; NA17; PAX3; ALK; Androgen receptor (AR); Cyclin B1; MYCN; RhoC; tyrosine related protein 2 (TRP-2); GD3; Fucosyl GM1; PSCA; sLe (a); CYP1B1; PLCA1; GM3; BORIS; Tn; GloboH; Ets variant gene 6/acute myeloid leukemia 1 gene ETS (ETV6-AML); NY-BR-1; RGS5; squamous antigen rejecting tumor or 3 (SART3); STn; Carbonic anhydrase IX; PAX5; OY-TES1; Sperm protein 17; LCK; HMWMAA; AKAP-4; SSX2; B7H3; Legumain; Tie 2; Page 4; VEGFR2; MAD-CT-1; FAP; PDGFR; MAD-CT-2; Fos-related antigen 1; TAG-72; 9D7; EphA3; Telomerase; SAP-1; BAGE family; CAGE family; GAGE family; MAGE family; SAGE family; XAGE family; preferentially expressed antigen of melanoma (PRAME); melanocortin 1 receptor (MC1R); β-catenin; BRCA1/2; CDK4; chronic myelogenous leukemia 66 (CML66); and TGF-β. In certain embodiments, the host self-antigens include prostatic acidic phosphatase, Wilms tumor suppressor protein, mesothelin, or Her-2.

In some embodiments, the methods are directed to the prevention or treatment of cancer. The cancer includes, but is not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrim; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia; and Wilms tumor (kidney cancer).

In some embodiments, the methods are directed to the treatment or prevention of tumor virus positive cancer. In some embodiments, the methods are directed to a method of generating an immune response to a tumor-associated antigen in a subject.

In some embodiments, the methods of the present disclosure provide administration of a CMV vector that does not express an active UL128, UL130, UL146, and UL147 protein due to the presence of a mutation in the nucleic acid sequence encoding UL128, UL130, UL146, and UL147 or homologs thereof, or orthologs thereof (homologous genes of CMV that infect other species). In some other embodiments the vector does not express an active UL128, UL130, UL146, UL147, and US11 protein due to the presence of a mutation in the nucleic acid sequence encoding UL128, UL130, UL146, UL147, and US11 or homologs thereof, or orthologs thereof (homologous genes of CMV that infect other species). The mutation may be any mutation that results in a lack of expression of the active UL128, UL130, UL146, UL147, or US11 proteins. Such mutations may include point mutations, frameshift mutations, deletions of less than all of the sequence that encodes the protein (truncation mutations), or deletions of all of the nucleic acid sequence that encodes the protein, or any other mutations. Exemplary vectors are described in U.S. Pat. Nos. 9,783,823 and 9,862,972, and US Appl. Pub. No. 2018/0298404, which are herein incorporated by reference.

In further examples, the CMV vector does not express an active UL128, UL130, UL146, and UL147 or the vector does not express an active UL128, UL130, UL146, UL147, and US11 protein due to the presence of a nucleic acid sequence in the vector that comprises an antisense or RNAi sequence (siRNA or miRNA) that inhibits the expression of the UL128, UL130, UL146, UL147, or US11 protein. Mutations and/or antisense and/or RNAi may be used in any combination to generate a CMV vector lacking active UL128, UL130, UL146, UL147, or US11.

In some embodiments, the CD8+ T cell response elicited by the vector is characterized by having at least 10% of the CD8+ T cells directed against epitopes presented by MHC-E. In further examples, at least 15%, at least 20%, at least 30%, at least 40%, at least 50%, at least 60%, at least 75%, at least 90%, at least 95%, or at least 95% of the CD8+ T cells are restricted by MIC-E. In some embodiments, the CD8+ T cells restricted by MHC-E recognize peptides shared by at least 90% of other subjects immunized with the vector. In some embodiments, the CD8+ T cells are directed against a supertope presented by MHC-E. In some embodiments, the CD8+ T cell response elicited by this vector is characterized by having at least 10% of the CD8+ T cells directed against epitopes presented by MHC-II. In further examples, at least 15%, at least 20%, at least 30%, at least 40%, at least 50%, at least 60%, at least 75%, at least 90%, at least 95% or at least 95% of the CD8+ T cells are restricted by MHC-II. In some embodiments, the CD8+ T cells restricted by MHC-II recognize peptides shared by at least 90% of other subjects immunized with the vector. In some embodiments, the CD8+ T cells are directed against a supertope presented by MHC-II.

In some embodiments, the method further comprises identifying a CD8+ T cell receptor from the CD8+ T cells elicited either by a UL128-130 and UL146-147-deleted HCMV vector in humans, or the RhCMV strain 68-1 vector in rhesus macaques, or UL128-130 and UL146-147-deleted CyCMV vectors in cynomolgus macaques. In some embodiments, the CD8+ T cell receptor is identified by RNA or DNA sequencing. In some embodiments, the method further comprises a CD8+ T cell receptor that recognizes MHC-E or MHC-II supertopes. In some embodiments, the MHC-E supertopes comprise peptides derived from PAP, WT1, MSLN, HER2, HPV E6 and E7 from strain 16, HPV E6 and E7 from strain 18, or HPV E6/E7 fusion proteins.

In some embodiments, the MHC-E or MHC-II supertope peptides have at least 50%, at least 60%, at least 70%, at least 75%, at least 80%, at least 85%, at least 90%, or at least 95%, at least 96%, at least 97%, at least 98%, at least 99%, or are 100% identical to the amino acid sequence corresponding to PAP (ARAASLSLGFLFLLF) (SEQ ID NO: 2); (KELKFVTLVFRHGDR) (SEQ ID NO: 3); (QLTQLGMEQHYELGE) (SEQ ID NO: 4); (LNESYKHEQVYIRST) (SEQ ID NO: 5); (NHMKRATQMPSYKKL) (SEQ ID NO: 6); or (MVLLFIHIRRGPCWQ) (SEQ ID NO: 7). In some embodiments, the MHC-E or MHC-II supertope peptides have at least 50%, at least 60%, at least 70%, at least 75%, at least 80%, at least 85%, at least 90%, or at least 95% identity to the amino acid sequence corresponding to WT1 (VPEPASQHTLRSGPG) (SEQ ID NO: 8); (SAERLQGRRSRGASG) (SEQ ID NO: 9); LQGRRSRGASGSEPQ (SEQ ID NO: 13); or HEDPMGQQGSLGEQQ (SEQ ID NO: 14). In some embodiments, the MHC-E or MHC-II supertope peptides have at least 50%, at least 60%, at least 70%, at least 75%, at least 80%, at least 85%, at least 90%, or at least 95%, at least 96%, at least 97%, at least 98%, at least 99%, or are 100% identical to the amino acid sequence corresponding to MSLN (IDESLIFYKKWELEA) (SEQ ID NO: 10); (PFTYEQLDVLKHKLD) (SEQ ID NO: 11); or (FMKLRTDAVLPLTVA) (SEQ ID NO: 12).

In some embodiments, the method further comprises identifying a CD8+ T cell receptor from the CD8+ T cells elicited either by a UL128-130 and UL146-147-deleted HCMV vector in humans, or the RhCMV strain 68-1 vector in rhesus macaques, or UL128-130 and UL146-147-deleted CyCMV vectors in cynomolgus macaques, wherein the CD8+ T cell receptor recognizes a MHC-II/heterologous antigen-derived peptide complex. In some embodiments, the CD8+ T cell receptor is identified by RNA or DNA sequencing.

In some embodiments, the method further comprises a CD8+ T cell receptor that recognizes specific MHC-II supertopes. In some embodiments, the specific MHC-II supertopes comprise peptides derived from PAP, WT1, MSLN, HER2, HPV E6 and E7 from strain 16, HPV E6 and E7 from strain 18, or HPV E6/E7 fusion proteins.

In some embodiments, the recombinant CMV vector expresses an active UL128 and UL130, and an inactive US11. In some embodiments, the CD8+ T cell response elicited by this vector is characterized by having at least 10% of the CD8+ T cells directed against epitopes presented by MHC-I. In further examples, at least 15%, at least 20%, at least 30%, at least 40%, at least 50%, at least 60%, at least 75%, at least 90%, at least 95%, or at least 95% of the CD8+ T cells are restricted by MHC-I.

In some embodiments, the method further comprises identifying a CD8+ T cell receptor from the CD8+ T cells elicited by the CMV vector, wherein the CD8+ T cell receptor recognizes a MHC-I/heterologous antigen-derived peptide complex. In some embodiments, the T cell receptor is from human or monkey T cells. In some embodiments, the CD8+ T cell receptor is identified by RNA or DNA sequencing.

In some embodiments, the recombinant CMV vector is administered to prevent or treat cancer. In some embodiments the cancer is acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, mesothelioma, malignant mesothelioma, kidney cancer, cervical cancer, oropharyngeal cancer, anal cancer, penile cancer, vaginal cancer, vulvar cancer, breast cancer, lung cancer, ovarian cancer, prostate cancer, pancreatic cancer, colon cancer, renal cell carcinoma, or germ cell tumors.

In some embodiments, the recombinant CMV vector is administered to prevent or treat tumor virus positive cancer. In some embodiments, the tumor virus positive cancer is acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, mesothelioma, malignant mesothelioma, kidney cancer, cervical cancer, oropharyngeal cancer, anal cancer, penile cancer, vaginal cancer, vulvar cancer, breast cancer, lung cancer, ovarian cancer, prostate cancer, pancreatic cancer, colon cancer, renal cell carcinoma, or germ cell tumors.

The term tumor virus, includes but is not limited to, human T-lymphotropic virus, herpes simplex virus, hepatitis B virus, hepatitis C virus, human papillomavirus (HPV), human polyomavirus, Kaposi's sarcoma-associated herpesvirus, merkel cell polyomavirus, and Epstein-barr virus. In some embodiments the tumor virus antigen is E6 and E7 from HPV strain 16 or E6 and E7 from HPV strain 18. In some embodiments the tumor virus antigen is a fusion of E6 and E7 from HPV. The tumor virus antigen may be a protein derived from any portion of the tumor virus. For example, in some embodiments, the tumor virus antigen may be derived from the core, envelope, surface, or polymerase proteins.

Also disclosed herein are methods of generating an immune response to at least one tumor-associated antigen in a subject. The method involves administering an effective amount of a recombinant CMV vector comprising the at least one tumor-associated antigen to the subject. In some embodiments, the CMV vector is characterized by having a nucleic acid sequence that does not express active UL128, UL130, or US11 proteins.

In some embodiments, the vector does not express: an active UL128, UL130, or US11 protein due to the presence of a mutation in the nucleic acid sequence encoding UL128, UL130, or US11 or homologs thereof, or orthologs thereof (homologous genes of CMV that infect other species). The mutation may be any mutation that results in a lack of expression of active proteins. Such mutations may include point mutations, frameshift mutations, deletions of less than all of the sequence that encodes the protein (truncation mutations), or deletions of all of the nucleic acid sequence that encodes the protein, or any other mutations.

In some further examples, the vector does not express an active UL128, UL130, or US11 protein due to the presence of a nucleic acid sequence in the vector that comprises an antisense or RNAi sequence (siRNA or miRNA) that inhibits the expression of the UL128, UL130, or US11 protein. Mutations and/or antisense and/or RNAi may be used in any combination to generate a CMV vector lacking active UL128, UL130, or US11.

In some embodiments, the method further comprises identifying a CD8+ T cell receptor from the CD8+ T cells elicited by the CMV vector, wherein the CD8+ T cell receptor recognizes a MHC-I heterologous antigen-derived peptide complex. In some embodiments, the CD8+ T cell receptor is identified by RNA or DNA sequencing.

Also disclosed herein is a method of generating CD8+ T cells that recognize MHC-E-peptide complexes. This method involves administering to a first subject (or animal) a CMV vector in an amount effective to generate a set of CD8+ T cells that recognize MHC-E/peptide complexes. The CMV vector comprises a first nucleic acid sequence encoding at least one heterologous antigen and does not express: an active UL128 protein or ortholog thereof, an active UL130 protein or ortholog thereof, or an active UL146 protein or ortholog thereof, an active UL147 protein or ortholog thereof. The antigen may be any antigen, including a pathogen-specific antigen, a tumor virus antigen, a tumor-associated antigen, or a host self-antigen. In some embodiments, the host self-antigen is an antigen derived from the variable region of a T cell receptor or a B cell receptor.

In some embodiments, the tumor associated antigens have at least 50%, at least 60%, at least 70%, at least 75%, at least 80%, at least 85%, at least 90%, or at least 95%, at least 96%, at least 97%, at least 98%, at least 99%, or are 100% identical to the amino acid sequence corresponding to PAP (ARAASLSLGFLFLLF) (SEQ ID NO: 2); (KELKFVTLVFRHGDR) (SEQ ID NO: 3); (QLTQLGMEQHYELGE) (SEQ ID NO: 4); (LNESYKHEQVYIRST) (SEQ ID NO: 5); (NHMKRATQMPSYKKL) (SEQ ID NO: 6); or (MVLLFIHIRRGPCWQ) (SEQ ID NO: 7). In some embodiments, the tumor associated antigens have at least 50%, at least 60%, at least 70%, at least 75%, at least 80%, at least 85%, at least 90%, or at least 95% identity to the amino acid sequence corresponding to WT1 (VPEPASQHTLRSGPG) (SEQ ID NO: 8); (SAERLQGRRSRGASG) (SEQ ID NO: 9); LQGRRSRGASGSEPQ (SEQ ID NO: 13); or HEDPMGQQGSLGEQQ (SEQ ID NO: 14). In some embodiments, the tumor associated antigens have at least 50%, at least 60%, at least 70%, at least 75%, at least 80%, at least 85%, at least 90%, or at least 95%, at least 96%, at least 97%, at least 98%, at least 99%, or are 100% identical to the amino acid sequence corresponding to MSLN (IDESLIFYKKWELEA) (SEQ ID NO: 10); (PFTYEQLDVLKHKLD) (SEQ ID NO: 11); or (FMKLRTDAVLPLTVA) (SEQ ID NO: 12).

This method further comprises: administering to a first subject a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof. In some embodiments, the method may further comprise identifying a first CD8+ TCR from the first set of CD8+ T cells, wherein the first CD8+ TCR recognizes a MHC-E/tumor-associated antigen-derived peptide complex. In some embodiments, the method may further comprise isolating a second set of one or more CD8+ T cells from a second subject. In some embodiments, the method may further comprise transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-E/tumor-associated antigen peptide complexes tumor-associated antigen.

The method further comprises: isolating from a first subject a first set of CD8+ T cells, wherein the first subject has been administered a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof. In some embodiments, the method further comprises identifying a first CD8+ TCR from the first set of CD8+ T cells, wherein the first CD8+ TCR recognizes a MHC-E/tumor-associated antigen-derived peptide complex. In some embodiments, the method further comprises isolating a second set of one or more CD8+ T cells from a second subject. In some embodiments, the method further comprises transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-E/tumor-associated antigen peptide complexes.

The one or more CD8+ T cells for transfection with the expression vector may be isolated from the first subject or a second subject.

In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

In some embodiments, the method further comprises identifying a CD8+ T cell receptor from the CD8+ T cells elicited by the CMV vector, wherein the CD8+ T cell receptor recognizes a MHC-E/heterologous antigen-derived peptide complex. In some embodiments, the CD8+ T cell receptor is identified by RNA or DNA sequencing. In some embodiments, the method further comprises a CD8+ T cell receptor that recognizes MHC-E supertopes. In some embodiments, the MHC-E supertopes comprise peptides derived from one or more of PAP, WT1, MSLN, HER2, and E6 or E7 of HPV16 or HPV18.

Also disclosed is a transfected CD8+ T cell that recognizes MHC-E-peptide complexes prepared by a process comprising the steps of: (a) administering to a first subject a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-E/tumor-associated antigen-derived peptide complex; (c) isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-E/tumor-associated antigen peptide complexes tumor-associated antigen.

Also disclosed is a transfected CD8+ T cell that recognizes MHC-E-peptide complexes prepared by a process comprising the steps of: (a) isolating from a first subject a first set of CD8+ T cells, wherein the first subject has been administered a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-E/tumor-associated antigen-derived peptide complex; (c) isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-E/tumor-associated antigen peptide complexes.

The CMV vector comprises a first nucleic acid sequence encoding at least one heterologous antigen and does not express: an active UL128 protein or ortholog thereof, an active UL130 protein or ortholog thereof, an active UL146 protein or ortholog thereof, or an active UL147 protein or ortholog thereof. The expression vector comprises a nucleic acid sequence encoding a second CD8+ T cell receptor and a promoter operably linked to the nucleic acid sequence encoding the second CD8+ T cell receptor, wherein the second CD8+ T cell receptor comprises CDR3α and CDR3β of the first CD8+ T cell receptor. The heterologous antigen may be any antigen, including a pathogen-specific antigen, an tumor virus antigen, or a host self-antigen.

In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

In some embodiments, the first CD8+ T cell receptor is identified by RNA or DNA sequencing.

Also disclosed herein are methods of treating a disease, such as cancer, a pathogenic infection, or an immune disease or disorder, the method comprising administering the transfected T cell that recognizes MHC-E peptide complexes to the first or second subject. Also disclosed herein are methods of inducing an immune response to a host self-antigen or tissue-specific antigen, the method comprising administering the transfected T cell that recognizes MHC-E-peptide complexes to the first or second subject. Also disclosed herein is the use of the CD8+ T cell in the manufacture of a medicament for the treatment or prevention of cancer. Also disclosed herein, is the use of the CD8+ T cell in the manufacture of a medicament for inducing an immune response to a host self-antigen in a subject.

Also disclosed herein is a method of generating CD8+ T cells that recognize MHC-II-peptide complexes. This method involves administering to a first subject a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof. In some embodiments, the method may further comprise identifying a first CD8+ TCR from the first set of CD8+ T cells, wherein the first CD8+ TCR recognizes a MHC-II/tumor antigen-derived peptide complex. In some embodiments, the method may further comprise isolating a second set of one or more CD8+ T cells from a second subject. In some embodiments, the method may further comprise transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-II/tumor antigen peptide complexes.

In some embodiments, the method involves isolating from a first subject a first set of CD8+ T cells, wherein the first subject has been administered a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof. In some embodiments, the method further comprises identifying a first CD8+ TCR from the first set of CD8+ T cells, wherein the first CD8+ TCR recognizes a MHC-II/tumor antigen-derived peptide complex. In some embodiments, the method further comprises isolating a second set of one or more CD8+ T cells from a second subject. In some embodiments, the method further comprises transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-II/tumor antigen peptide complexes.

The CMV vector comprises a first nucleic acid sequence encoding at least one heterologous antigen and does not express: an active UL128 protein or ortholog thereof, an active UL130 protein or ortholog thereof, an active UL146 protein or ortholog thereof, or an active UL147 protein or ortholog thereof protein or ortholog thereof. The antigen may be any antigen, including a pathogen-specific antigen, an tumor virus antigen, a tumor-associated antigen, a tissue specific antigen, or a host self-antigen. In some embodiments, the host self-antigen is an antigen derived from the variable region of a T cell receptor or a B cell receptor. In some embodiments, the host self-antigen may be a tumor-associated antigen (TAA) that is abnormally expressed by cancer cells. TAAs include, but are not limited to, i) germline/testis antigens expressed in cancer cells, ii) cell lineage differentiation antigens not expressed in adult tissue, or iii) antigens overexpressed in cancer cells. In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

In some embodiments, the method further comprises identifying a CD8+ T cell receptor from the CD8+ T cells elicited by the CMV vector, wherein the CD8+ T cell receptor recognizes a MHC-II/heterologous antigen-derived peptide complex. In some embodiments, the CD8+ T cell receptor is identified by RNA or DNA sequencing.

In some embodiments, the method further comprises a CD8+ T cell receptor that recognizes specific MHC-II supertopes. In some embodiments, the specific MHC-II supertopes comprise peptides derived from one or more of PAP, WT1, MSLN, HER2, HPV E6 or E7 from strain 16, HPV E6 or E7 from strain 18, and HPV E6/E7 fusion proteins.

Also disclosed is a transfected CD8+ T cell that recognizes MHC-II-peptide complexes prepared by a process comprising the steps of: (a) administering to a first subject a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-II/tumor antigen-derived peptide complex; (c)isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-II/tumor antigen peptide complexes.

Also disclosed is a transfected CD8+ T cell that recognizes MHC-II-peptide complexes prepared by a process comprising the steps of: (a) isolating from a first subject a first set of CD8+ T cells, wherein the first subject has been administered a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof, (b) identifying a first CD8+ TCR from the first set of CD8+ T cells, wherein the first CD8+ TCR recognizes a MHC-II/tumor antigen-derived peptide complex; (c) isolating a second set of one or more CD8+ T cells from a second subject; and (d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-II/tumor antigen peptide complexes.

In some embodiments, the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

The CMV vector comprises a first nucleic acid sequence encoding at least one heterologous antigen and does not express: an active UL128 protein or ortholog thereof, an active UL130 protein or ortholog thereof, and an active UL146 protein or ortholog thereof, an active UL147 protein or ortholog thereof. The expression vector comprises a nucleic acid sequence encoding a second CD8+ T cell receptor and a promoter operably linked to the nucleic acid sequence encoding the second CD8+ T cell receptor, wherein the second CD8+ T cell receptor comprises CDR3α and CDR3β of the first CD8+ T cell receptor. The heterologous antigen may be any antigen, including a pathogen-specific antigen, a tumor virus antigen, tissue-specific antigen, or a host self-antigen. In some embodiments, the first CD8+ T cell receptor is identified by RNA or DNA sequencing. Also disclosed herein are methods of treating a disease, such as cancer, a pathogenic infection, or an immune disease or disorder, the method comprising administering the transfected T cell that recognizes MHC-II peptide complexes to the first or second subject. Also disclosed herein are methods of inducing an immune response to a host self-antigen or tissue-specific antigen, the method comprising administering the transfected T cell that recognizes MHC-II-peptide complexes to the first or second subject.

Also disclosed herein is a method of generating CD8+ T cells that recognize MHC-I-peptide complexes. This method involves administering to a first subject (or animal) a CMV vector in an amount effective to generate a set of CD8+ T cells that recognize MHC-I/peptide complexes. The CMV vector comprises a first nucleic acid sequence encoding at least one heterologous antigen and expresses an active UL128 protein or ortholog thereof, an active UL130 protein or ortholog thereof, an active UL146 protein or ortholog thereof, and an active UL147 protein or ortholog thereof. The antigen may be any antigen, including a pathogen-specific antigen, a tumor virus antigen, a tumor-associated antigen, a tissue specific antigen, or a host self-antigen. In some embodiments, the host self-antigen is an antigen derived from the variable region of a T cell receptor or a B cell receptor.

In some embodiments, the method further comprises identifying a CD8+ T cell receptor from the CD8+ T cells elicited by the CMV vector, wherein the CD8+ T cell receptor recognizes a MHC-I/heterologous antigen-derived peptide complex. In some embodiments, the CD8+ T cell receptor is identified by RNA or DNA sequencing.

In some embodiments, the method further comprises a CD8+ T cell receptor that recognizes specific MHC-I epitopes. In some embodiments, the specific MHC-I epitopes comprise one or more of PAP, WT1, MSLN, HER2, HPV E6 or E7 from strain 16, HPV E6 or E7 from strain 18, and HPV E6/E7 fusion proteins.

In some embodiments, this method may further comprise administering the one or more transfected T cells to the first or second subject to treat a disease, such as cancer, a pathogenic infection, or an immune disease or disorder. In some embodiments, this method may further comprise administering the one or more transfected T cells to the first or second subject to induce an immune response to a tumor-associated antigen.

The CMV vectors disclosed herein may be used as an immunogenic, immunological, or vaccine composition containing the recombinant CMV virus or vector, and a pharmaceutically acceptable carrier or diluent. An immunological composition containing the recombinant CMV virus or vector (or an expression product thereof) elicits an immunological response—local or systemic. The response can, but need not be, protective. An immunogenic composition containing the recombinant CMV virus or vector (or an expression product thereof) likewise elicits a local or systemic immunological response which can, but need not be, protective. A vaccine composition elicits a local or systemic protective response. Accordingly, the terms “immunological composition” and “immunogenic composition” include a “vaccine composition” (as the two former terms may be protective compositions).

The recombinant CMV vectors disclosed herein may be human cytomegalovirus vectors, rhesus macaque cytomegalovirus vectors, or cynomolgus macaque vectors.

The recombinant CMV vectors disclosed herein may be used in methods of inducing an immunological response in a subject comprising administering to the subject an immunogenic, immunological, or vaccine composition comprising the recombinant CMV virus or vector and a pharmaceutically acceptable carrier or diluent.

The recombinant CMV vectors disclosed herein may be used in therapeutic compositions containing the recombinant CMV virus or vector and a pharmaceutically acceptable carrier or diluent. The CMV vectors disclosed herein may be prepared by inserting DNA comprising a sequence that encodes the tumor-associated antigen into an essential or non-essential region of the CMV genome. The method may further comprise deleting one or more regions from the CMV genome. The method may comprise in vivo recombination. Thus, the method may comprise transfecting a cell with CMV DNA in a cell-compatible medium in the presence of donor DNA comprising the heterologous DNA flanked by DNA sequences homologous with portions of the CMV genome, whereby the heterologous DNA is introduced into the genome of the CMV, and optionally then recovering CMV modified by the in vivo recombination. The method may also comprise cleaving CMV DNA to obtain cleaved CMV DNA, ligating the heterologous DNA to the cleaved CMV DNA to obtain hybrid CMV-heterologous DNA, transfecting a cell with the hybrid CMV-heterologous DNA, and optionally then recovering CMV modified by the presence of the heterologous DNA Since in vivo recombination is comprehended, the method accordingly also provides a plasmid comprising donor DNA not naturally occurring in CMV encoding a polypeptide foreign to CMV, the donor DNA is within a segment of CMV DNA that would otherwise be co-linear with an essential or non-essential region of the CMV genome such that DNA from an essential or nonessential region of CMV is flanking the donor DNA The heterologous DNA may be inserted into CMV to generate the recombinant CMV in any orientation that yields stable integration of that DNA, and expression thereof, when desired.

The DNA encoding the heterologous antigen in the recombinant CMV vector may also include a promoter. The promoter may be from any source such as a herpes virus, including an endogenous cytomegalovirus (CMV) promoter, such as a human CMV (HCMV), rhesus macaque CMV (RhCMV), murine, or other CMV promoter. The promoter may also be a nonviral promoter such as the EF1α promoter. The promoter may be a truncated transcriptionally active promoter which comprises a region transactivated with a transactivating protein provided by the virus and the minimal promoter region of the full-length promoter from which the truncated transcriptionally active promoter is derived. The promoter may be composed of an association of DNA sequences corresponding to the minimal promoter and upstream regulatory sequences. A minimal promoter is composed of the CAP site plus ATA box (minimum sequences for basic level of transcription; unregulated level of transcription); “upstream regulatory sequences” are composed of the upstream element(s) and enhancer sequence(s). Further, the term “truncated” indicates that the full-length promoter is not completely present, i.e., that some portion of the full-length promoter has been removed. And, the truncated promoter may be derived from a herpesvirus such as MCMV or HCMV, e.g., HCMV-IE or MCMV-IE. There may be up to a 40% and even up to a 90% reduction in size, from a full-length promoter, based upon base pairs. The promoter may also be a modified nonviral promoter. As to HCMV promoters, reference is made to U.S. Pat. Nos. 5,168,062 and 5,385,839. As to transfecting cells with plasmid DNA for expression therefrom, reference is made to Feigner et al. (1994), J Biol. Chem. 269, 2550-2561. And, as to direct injection of plasmid DNA as a simple and effective method of vaccination against a variety of infectious diseases reference is made to Science, 259:1745-49, 1993. It is therefore within the scope of this disclosure that the vector may be used by the direct injection of vector DNA.

Also disclosed is an expression cassette that may be inserted into a recombinant virus or plasmid comprising the truncated transcriptionally active promoter. The expression cassette may further include a functional truncated polyadenylation signal; for instance an SV40 polyadenylation signal which is truncated, yet functional. Considering that nature provided a larger signal, it is indeed surprising that a truncated polyadenylation signal is functional. A truncated polyadenylation signal addresses the insert size limit problems of recombinant viruses such as CMV. The expression cassette may also include heterologous DNA with respect to the virus or system into which it is inserted; and that DNA may be heterologous DNA as described herein.

As to antigens for use in vaccine or immunological compositions, see also Stedman's Medical Dictionary (24th edition, 1982, e.g., definition of vaccine (for a list of antigens used in vaccine formulations); such antigens or epitopes of interest from those antigens may be used. As to tumor-associated antigens, one skilled in the art may select a tumor-associated antigen and the coding DNA therefor from the knowledge of the amino acid and corresponding DNA sequences of the peptide or polypeptide, as well as from the nature of particular amino acids (e.g., size, charge, etc.) and the codon dictionary, without undue experimentation.

One method to determine T epitopes of an antigen involves epitope mapping. Overlapping peptides of the tumor-associated antigen are generated by oligo-peptide synthesis. The individual peptides are then tested for their ability to induce T cell activation. This approach has been particularly useful in mapping T cell epitopes since the T cell recognizes short linear peptides complexed with MHC molecules.

An immune response to a tumor-associated antigen is generated, in general, as follows: T cells recognize proteins only when the protein has been cleaved into smaller peptides and is presented in a complex called the “major histocompatibility complex (MHC)” located on another cell's surface. There are two classes of MHC complexes—class I and class II, and each class is made up of many different alleles. Different species, and individual subjects have different types of MHC complex alleles; they are said to have a different MHC type. One type of MHC class I molecule is called MHC-E (HLA-E in humans, Mamu-E in RM, Qa-lb in mice). Unlike other MHC-I molecules, MHC-E is highly conserved within and between mammalian species.

It is noted that the DNA comprising the sequence encoding the tumor-associated antigen may itself include a promoter for driving expression in the CMV vector or the DNA may be limited to the coding DNA of the tumor-associated antigen. This construct may be placed in such an orientation relative to an endogenous CMV promoter that it is operably linked to the promoter and is thereby expressed. Further, multiple copies of DNA encoding the tumor-associated antigen or use of a strong or early promoter or early and late promoter, or any combination thereof, may be done so as to amplify or increase expression. Thus, the DNA encoding the tumor-associated antigen may be suitably positioned with respect to a CMV endogenous promoter, or those promoters may be translocated to be inserted at another location together with the DNA encoding the tumor-associated antigen. Nucleic acids encoding more than one tumor-associated antigen may be packaged in the CMV vector.

Further disclosed are pharmaceutical and other compositions containing the disclosed CMV vectors. Such pharmaceutical and other compositions may be formulated so as to be used in any administration procedure known in the art. Such pharmaceutical compositions may be via a parenteral route (intradermal, intramuscular, subcutaneous, intravenous, or others). The administration may also be via a mucosal route, e.g., oral, nasal, genital, etc.

The disclosed pharmaceutical compositions may be prepared in accordance with standard techniques well known to those skilled in the pharmaceutical arts. Such compositions may be administered in dosages and by techniques well known to those skilled in the medical arts taking into consideration such factors as the breed or species, age, sex, weight, and condition of the particular patient, and the route of administration. The compositions may be administered alone, or may be co-administered or sequentially administered with other CMV vectors or with other immunological, antigenic or vaccine or therapeutic compositions. Such other compositions may include purified native antigens or epitopes or antigens or epitopes from the expression by a recombinant CMV or another vector system; and are administered taking into account the aforementioned factors.

Examples of compositions include liquid preparations for orifice, e.g., oral, nasal, anal, genital, e.g., vaginal, etc., administration such as suspensions, syrups or elixirs; and, preparations for parenteral, subcutaneous, intradermal, intramuscular, or intravenous administration (e.g., injectable administration) such as sterile suspensions or emulsions. In such compositions the recombinant may be in admixture with a suitable carrier, diluent, or excipient such as sterile water, physiological saline, glucose, or the like.

Antigenic, immunological, or vaccine compositions typically may contain an adjuvant and an amount of the CMV vector or expression product to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryllipid A, phospholipid conjugates such as those described by Goodman-Snitkoff et al., J Immunol. 147:410-415 (1991), encapsulation of the protein within a proteoliposome as described by Miller et al., J Exp. Med. 176:1739-1744 (1992), and encapsulation of the protein in lipid vesicles such as Novasome lipid vesicles (Micro Vescular Systems, Inc., Nashua, N.H.) may also be used.

The composition may be packaged in a single dosage form for immunization by parenteral (e.g., intramuscular, intradermal or subcutaneous) administration or orifice administration, e.g., perlingual (e.g., oral), intragastric, mucosal including intraoral, intraanal, intravaginal, and the like administration. And again, the effective dosage and route of administration are determined by the nature of the composition, by the nature of the expression product, by expression level if recombinant CMV is directly used, and by known factors, such as breed or species, age, sex, weight, condition, and nature of host, as well as LD50 and other screening procedures which are known and do not require undue experimentation. Dosages of expressed product may range from a few to a few hundred micrograms, e.g., 5 to 500 μg. The CMV vector may be administered in any suitable amount to achieve expression at these dosage levels. In nonlimiting examples: CMV vectors may be administered in an amount of at least 10² pfu; thus, CMV vectors may be administered in at least this amount; or in a range from about 10² pfu to about 10⁷ pfu. Other suitable carriers or diluents may be water or a buffered saline, with or without a preservative. The CMV vector may be lyophilized for resuspension at the time of administration or may be in solution.

It should be understood that the proteins and the nucleic acids encoding them of the present disclosure may differ from the exact sequences illustrated and described herein. Thus, the disclosure contemplates deletions, additions, truncations, and substitutions to the sequences shown, so long as the sequences function in accordance with the methods of the disclosure. In this regard, substitutions will generally be conservative in nature, i.e., those substitutions that take place within a family of amino acids. For example, amino acids are generally divided into four families: (1) acidic—aspartate and glutamate; (2) basic—lysine, arginine, and histidine; (3) nonpolar—alanine, valine, leucine, isoleucine, proline, phenylalanine, methionine, and tryptophan; and (4) uncharged polar—glycine, asparagine, glutamine, cysteine, serine threonine, and tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the proteins described but possessing minor amino acid substitutions that do not substantially affect the immunogenicity of the protein are, therefore, within the scope of the disclosure.

The nucleotide sequences of the present disclosure may be codon optimized, for example the codons may be optimized for use in human cells. For example, any viral or bacterial sequence may be so altered. Many viruses, including HIV and other lentiviruses, use a large number of rare codons and, by altering these codons to correspond to codons commonly used in the desired subject, enhanced expression of the tumor-associated antigen may be achieved as described in Andre et al., J Virol. 72:1497-1503, 1998.

Nucleotide sequences encoding functionally and/or antigenically equivalent variants and derivatives of the CMV vectors and the glycoproteins included therein are contemplated. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In some embodiments, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% homology or identity to the antigen, epitope, immunogen, peptide, or polypeptide of interest.

The various recombinant nucleotide sequences and antibodies and/or antigens of the disclosure are made using standard recombinant DNA and cloning techniques. Such techniques are well known to those of skill in the art. See for example, “Molecular Cloning: A Laboratory Manual”, second edition (Sambrook et al. 1989).

The CMV vectors described herein may contain mutations that may prevent host to host spread, thereby rendering the virus unable to infect immunocompromised or other subjects that could face complications as a result of CMV infection. The CMV vectors described herein may also contain mutations that result in the presentation of immunodominant and nonimmunodominant epitopes as well as non-canonical MHC restriction. However, mutations in the CMV vectors described herein do not affect the ability of the vector to reinfect a subject that has been previously infected with CMV. Such CMV mutations are described in, for example, US Patent Publications 2013-013676S; 2010-0142S23; 2014-014103S; and PCT application publication WO 2014/13S209, all of which are incorporated by reference herein.

The disclosed CMV vectors may be administered in vivo, for example where the aim is to produce an immunogenic response, including a CD8+ immune response, including an immune response characterized by a high percentage of the CD8+ T cell response being restricted by MHC-E, MHC-II, or MHC-I (or a homolog or ortholog thereof). For example, in some examples it may be desired to use the disclosed CMV vectors in a laboratory animal, such as rhesus macaques for preclinical testing of immunogenic compositions and vaccines using RhCMV. In other examples, it will be desirable to use the disclosed CMV vectors in human subjects, such as in clinical trials and for actual clinical use of the immunogenic compositions using HCMV.

For such in vivo applications the disclosed CMV vectors are administered as a component of an immunogenic composition further comprising a pharmaceutically acceptable carrier. In some embodiments, the immunogenic compositions of the disclosure are useful to stimulate an immune response against the heterologous antigen, including a tumor-associated antigen, a tumor virus antigen, or a host self-antigen and may be used as one or more components of a prophylactic or therapeutic vaccine against tumor-associated antigens, tumor virus antigens, or host self-antigens for the prevention, amelioration or treatment of cancer. The nucleic acids and vectors of the disclosure are particularly useful for providing genetic vaccines, i.e., vaccines for delivering the nucleic acids encoding the antigens of the disclosure to a subject, such as a human, such that the antigens are then expressed in the subject to elicit an immune response.

Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, [and is often 2, 4, 6 or 8 weeks. For humans, the interval is typically from 2 to 6 weeks. In a particularly advantageous embodiment of the present disclosure, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks, or 70 weeks. The immunization regimes typically have from 1 to 6 administrations of the immunogenic composition, but may have as few as one or two or four. The methods of inducing an immune response may also include administration of an adjuvant with the immunogens. In some instances, annual, biannual, or other long interval (5-10 years) booster immunization may supplement the initial immunization protocol. The present methods also include a variety of prime-boost regimens. In these methods, one or more priming immunizations are followed by one or more boosting immunizations. The actual immunogenic composition may be the same or different for each immunization and the type of immunogenic composition (e.g., containing protein or expression vector), the route, and formulation of the immunogens may also be varied. For example, if an expression vector is used for the priming and boosting steps, it may either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial, and viral expression vectors of the disclosure to provide priming and boosting regimens. CMV vectors may be used repeatedly while expressing different antigens derived from different pathogens.

### EXAMPLES

### Example 1: CMV Vaccines are Able to Overcome Immunological Tolerance

Rhesus cytomegalovirus (RhCMV) strain 68-1 based vaccine vectors elicit CD8+ T cell response that recognize SIV, TB, or malaria peptides in the context of MHC-E and MHC-II, instead of classical MHC-Ia molecules (Hansen et al. 2019. Cytomegalovirus vectors expressing Plasmodium knowlesi antigens induce immune responses that delay parasitemia upon sporozoite challenge. PLoS One 14:e0210252; Hansen et al., Science, 2013; Hansen et al., Science, 2016).

Several factors make HLA-E a particularly attractive target for cancer immunotherapy, including: (a) in a number of cancers it has been reported that cancer cells upregulate HLA-E (Kamiya 2019 J Clin Invest). Since HLA-E is a ligand for the inhibitory NKG2A receptor, this could be the result of a selection for cancer cells that evade an NK cell evasion; (b) normal tissue (with the exception of endothelial cells and some immune cells) express low levels of HLA-E; (c) cancer cells are often selected to express low levels of classical HLA molecules presumably as a means to escape T cell control; and (d) unlike classical HLA molecules, which are highly polymorphic, HLA-E is highly conserved. Thus, a HLA-E restricted TCR can be applied universally to transgenic T cells.

CMV-based vectors can be used in two ways as cancer immunotherapies: either directly as cancer vaccines in humans, or indirectly as vehicles to elicit MHC-E restricted TCRs in non-human primates. However, both approaches require the demonstration that: (a) CMV is capable of eliciting MHC-E-restricted CD8+ T cells to cancer antigens and (b) cancer cells are able to present cancer antigens. Since cancer antigens are often self-antigens, they are subject to immunological tolerance. Breaking immunological tolerance is challenging for most vector systems as exemplified by the fact that poxvectors (e.g., PROSTVAC) failed in clinical trials and likely needs to be combined with checkpoint-inhibitory antibodies to elicit effective prostate-antigen specific immune responses (https://www.onclive.com/web-exclusives/prostvac-misses-phase-iii-goal-in-prostate-cancer). Moreover, HLA-E is considered a highly selective receptor for the single peptide VL9 (itself derived from the signal peptide of polymorphic HLA molecules) and it is not known how commonly other peptides are loaded into HLA-E.

To evaluate the ability of CMV to elicit MHC-E restricted CD8+ T cell responses in the context of cancer antigens, strain 68.1 vectors expressing cancer antigens were generated and administered to rhesus macaques (RM). RhCMV strain 68-1 is deficient for the RhCMV homologs of UL128, UL130, UL146, and UL147. The vectors were designed to express one of the following inserts: 1) rhesus PAP, 2) human WT1 Ag, 3) human MSLN, 4) human HER2, and 5) HPV 16/18 E6+E7.

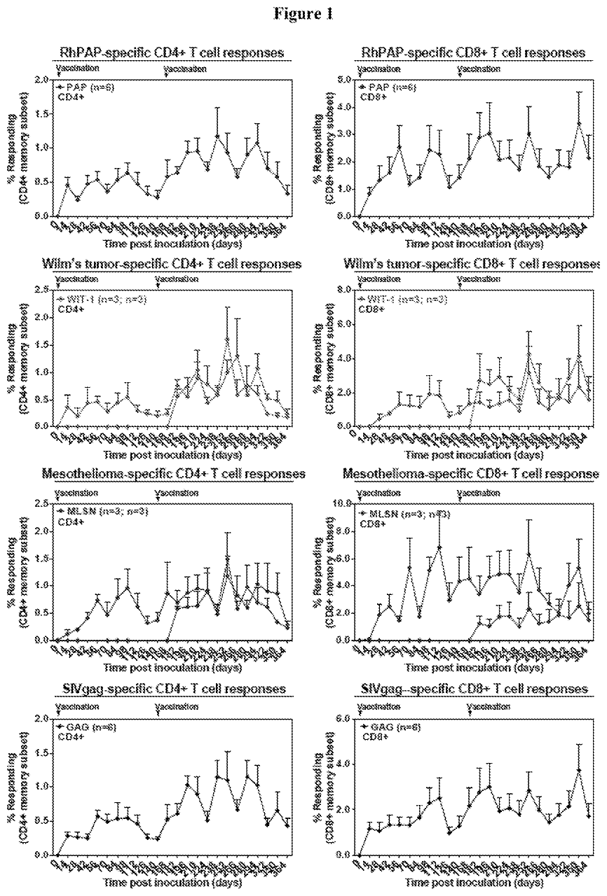

Six male RM were inoculated with RhCMV strain 68-1 expressing the cancer antigen prostatic acid phosphatase (PAP, rhesus) (68-1/PAP). As a control, the RM were also inoculated with the SIV gag antigen (68-1/SIVgag). Three of six RM were additionally co-inoculated with RhCMV strain 68-1 expressing the cancer antigen Wilms tumor suppressor protein (WT1, human) (68-1/WT1) whereas the other three RM were additionally co-inoculated with the RhCMV strain 68-1 expressing the cancer antigen mesothelin (MSLN, human) (68-1/MSLN). Each of the RM obtained a boosting immunization at day 140 and all monkeys received all antigens at this time point. CD4+ and CD8+ T cell responses were measured in peripheral blood mononuclear cells (PBMC) using overlapping peptide pools for each of the antigens by intracellular cytokine staining (ICS) at each of the indicated time points. Infected RM were able to elicit high frequency T cell responses to each of the antigens (FIG. 1). These results are highly significant since they demonstrate that CMV-based vaccines are able to overcome immunological tolerance and to elicit T cell responses to self-antigens as well as viral oncogenes. Importantly, negative side effects were not observed despite the fact that the T cells would be auto-reactive.

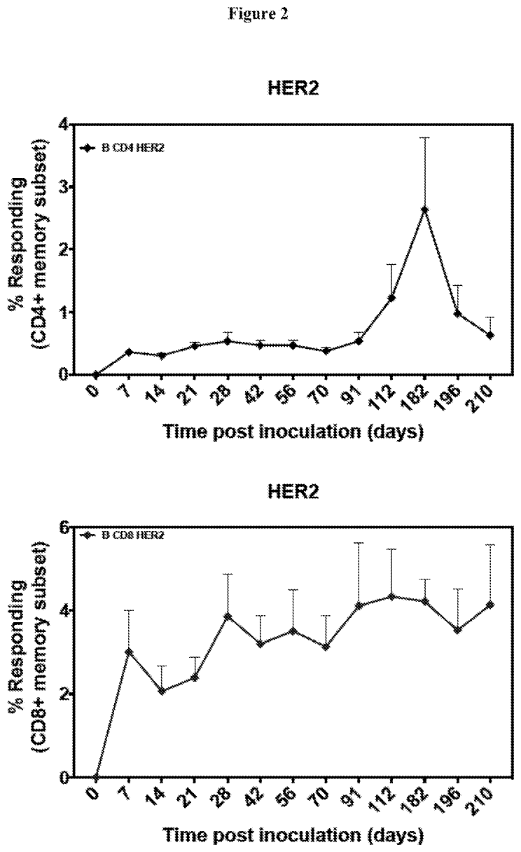

Next, six female RM were inoculated with RhCMV strain 68-1 expressing the cancer antigen HER2 (human) (68-1/HER2). CD4+ and CD8+ T cell responses were measured in PBMC using overlapping peptide pools for HER2 by ICS at each of the indicated time points. As shown in FIG. 2, the infected RM were able to elicit high frequency T cell responses to HER2 (FIG. 2).

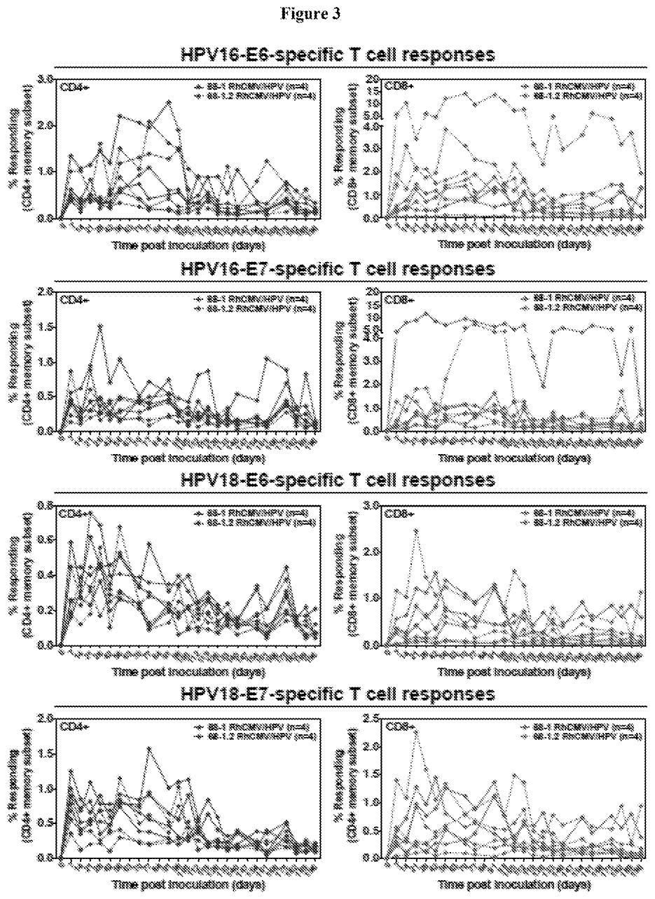

Four female RM were inoculated with RhCMV strain 68-1 (68-1/HPV) and four female RM were inoculated with RhCMV strain 68-1.2 (68-1.2/HPV) expressing a fusion protein of the E6 and E7 proteins of HPV16 and HPV18. RhCMV strain 68-1.2 is “repaired” for UL128-130, as described by Lilja A E and Shenk T, Proc Natl Acad Sci USA 105, 19950-19955 (2008). The vectors were designed to express one CD4+ and CD8+ T cell responses were measured in PBMC using overlapping peptide pools for HPV antigens by ICS at each of the indicated time points in FIG. 3. As shown in the FIG. 3, all inoculated RM were able to elicit high frequency T cell responses to HPV 16 and HPV18 E6 and E7 oncogenes. Strain 68-1 elicited CD8+ T cells restricted by MHC-II and MHC-E whereas strain 68-1.2 elicited CD8+ T cells restricted by MHC-I.

### Example 2: Cancer Cells can Present Cancer Antigens Via HLA-E

To further determine whether the T cells generated above are able to recognize cancer cells expressing these antigens, T cell responses were measured from CD8+ T cells incubated with K562 (human chronic myelogenic leukemia) cells expressing MHC-E. Since K562 cells do not express other MHC molecules, any peptide presentation to T cells would be mediated by MHC-E.

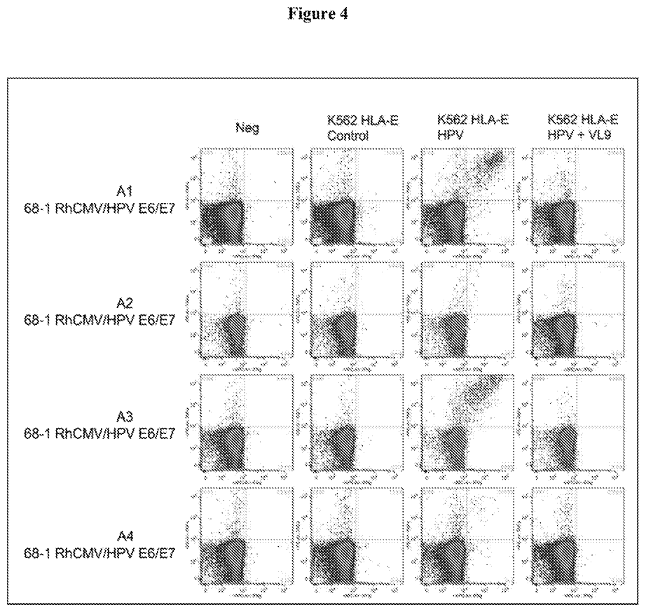

Four female RM were inoculated with 68-1 expressing a fusion protein of the E6 and E7 proteins of HPV16 and HPV18 (68-1/HPV). CD8+ T cells were isolated and co-incubated with K562 cells expressing either MHC-E or MHC-E and the same fusion protein of HPV. T cell responses were measured by intracellular cytokine staining for TNFα and IFNγ (FIG. 4). CD8+ T cells responding with both TNFα and IFNγ production appear in the upper right quadrant. MHC-E expressing K562 cells (K562-E) transfected with the HPV fusion protein were recognized by CD8+ T cells from two of the four RM that had been immunized with 68-1/HPV. Peptide presentation by MHC-E was further demonstrated by adding the peptide VMAPRTLLL (VL9) (SEQ ID NO: 1), which is a high affinity ligand for MHC-E. Addition of VL9 inhibited CD8+ T cell responses. This demonstrates that TCRs elicited in RM can recognize human MHC-E presenting peptides.

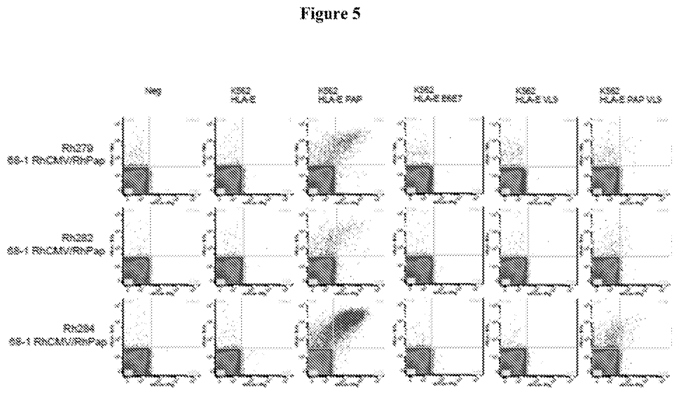

Next, it was determined whether the CMV vectors can generate T cells that are able to recognize cancer cells expressing host self-antigens, such as rhesus PAP. Three male RM were inoculated with 68-1/PAP. CD8+ T cells were isolated and co-incubated with K562 cells expressing MHC-E and K562 cells expressing MHC-E and PAP or MHC-E and the HPV E6-E7 fusion protein. T cell responses were measured by intracellular cytokine staining (ICS) for TNFα and IFNγ (FIG. 5). CD8+ T cells responding with both TNFα and IFNγ production appear in the upper right quadrant. PAP-specific MHC-E restricted CD8+ T cell responses were blocked with the MHC-E ligand peptide VMAPRTLLL (VL9) (SEQ ID NO: 1). K562-E transfected with PAP, but not K562-E cells transfected with the HPV fusion protein, were recognized by the CD8+ T isolated from RM immunized with 68-1/PAP, demonstrating specific recognition. Addition of VL9 inhibited the PAP-specific CD8+ T cell response demonstrating recognition was mediated by MHC-E.

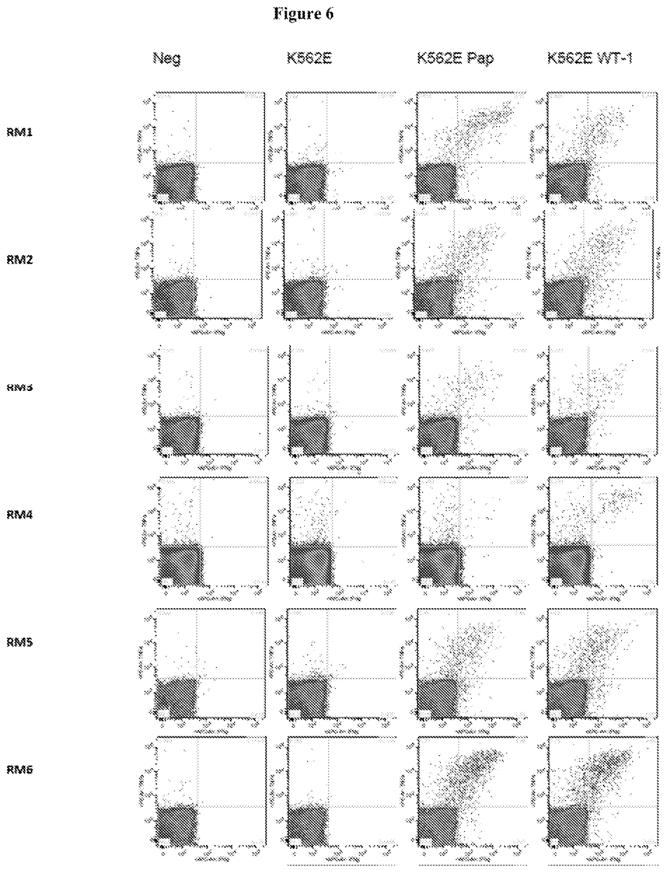

In a second experiment, six male RM were co-inoculated with 68-1/PAP and 68-1/WT1. CD8+ T cells were isolated and co-incubated with K562 cells expressing MHC-E and K562 cells expressing MHC-E and PAP (rhesus) or MHC-E and WT1 (human). T cell responses were measured by intracellular cytokine staining for TNFα and IFNγ (FIG. 6). CD8+ T cells responding with both TNFα and IFNγ production appear in the upper right quadrant. K562-E transfected with PAP or WT1 was recognized by CD8+ T cells from the six RM that were immunized with 68-1/PAP and 68-1/WT1.

In these experiments, cancer cells that express both MHC-E and a TAA are recognized by MHC-E restricted CD8+ T cells

The results demonstrate that CMV-based cancer vaccines overcome several of the challenges faced by immune tolerance, namely:

(i) anti-vector immunity does not affect the ability of CMV-vectors to elicit T cell responses to inserted antigens (Hansen et al., 2010. Evasion of CD8+ T cells is critical for superinfection by cytomegalovirus. Science 328:102-106); and

(ii) CMV-vectors elicit CD8+ T cells to TAAs that are similar in frequency than those elicited to foreign antigens. Thus CMV-based vaccines are exceptionally good at breaking immunological tolerance. Breaking immunological tolerance can occur in two ways: (a) induction of MHC-I restricted CD8+ T cells to non-canonical, MHC-I restricted epitopes (e.g., 68-1.2/PAP), or (b) induction of MHC-E and MHC-II restricted CD8+ T cells to unconventional epitopes (68-1-based vectors). This point is further illustrated by the observation that CMV-based vectors that lack US11 did not elicit CD8+ T cells. In contrast, US11-deletion results in vectors that elicit MHC-I restricted CD8+ T cells recognizing canonical, i.e., immunodominant, epitopes to non-self antigens (Hansen Science 2010, Hansen PlosONe 2019). The most likely explanation for the failure of US11-deleted vectors to elicit MHC-I restricted CD8+ T cells to self-antigens is that these T cells are eliminated in the thymus (=central tolerance). Central tolerance is likely why other vaccine and vector systems are inferior in eliciting CD8+ T cells to cancer antigens relative to CMV, because they are unable to elicit CD8+ T cells to non-canonical, or subdominant, MHC-I restricted epitopes and they are unable to elicit CD8+ T cells to MHC-II or MHC-E restricted epitopes. Since MHC-E is often upregulated in cancer cells, whereas MHC-I is downregulated, targeting MHC-E is likely to be particularly effective. There is currently no other vector system that can elicit MHC-E restricted CD8+ T cells to cancer antigens.

### Example 3: Identification of MHC-II and MHC-E Supertopes

Chimeric antigen receptor (CAR)-expressing transgenic T cells (CAR-T cells) have revolutionized the treatment of a number of cancers, particularly leukemias. In most cases, the CAR comprises an antibody-derived binding domain that recognizes a surface protein of the cancer cell (e.g., CD20 for B cell lymphomas). However, new CAR-T cells are generated for every patient in order to avoid rejection of the transgenic T cell, and as a result this treatment is extremely expensive. Off-the-shelf CAR-T cells that can be used in all patients are in development, but none have been approved for clinical use thus far.

Since CAR-T cells can eliminate all cells expressing a given antigen (e.g., all B cells express CD20) they can have the side effect of rendering the patient immunosuppressed or suffering from immune disease complications. Engineered TCR-T cells, i.e., T cells that are transgenic for a T cell receptor (TCR) recognizing a tumor-specific peptide in the context of MHC, offer another therapeutic approach, which may reduce such side effects. However, any given TCR will recognize only a specific MHC/peptide complex. Since the MHC is highly polymorphic, use of engineered TCRs is restricted to individuals that carry the correct MHC allele, a severe limitation of engineered TCR-T cells. In contrast, MHC-E is non-polymorphic among the human population so that a MHC-E-restricted TCR is “universal” (i.e., it can be used in every person). In fact, MHC-E is even conserved between non-human primates and humans so that TCRs elicited in RM recognize the human MHC-E and HLA-E/presenting peptides. MHC-E restricted TAA-specific TCRs generated in RM can thus be used to generate universal, off-the-shelf, human TCR T cells. A key step to identifying such T cells is the identification of MHC-E-restricted supertopes. With the identified Using supertope peptides, TCRs in activated T cells can then be identified.

It has been shown that deletion of the US11 homologue Rh189 elicits “canonical” MHC-I-restricted CD8+ T cell responses, i.e., peptides that are immunodominant in the context of conventional vaccines as a consequence of high affinity peptide binding to MHC-I and CD8+ T cells expressing T cell receptors with high affinity to the peptide/MHC-I complex. Taking advantage of this property, three different constructs were prepared that encode rhesus PAP and one of which does not encode active Rh189: 68-1.2/PAP (expected to elicit MHC-I restricted CD8+ T cell responses), 68-1/PAP (expected to elicit MHC-II and MHC-E restricted CD8+ T cell responses), and 68-1/PAPΔRh189 (expected to elicit MHC-II and MHC-E restricted C and canonical MHC-I restricted CD8+ T cell responses).

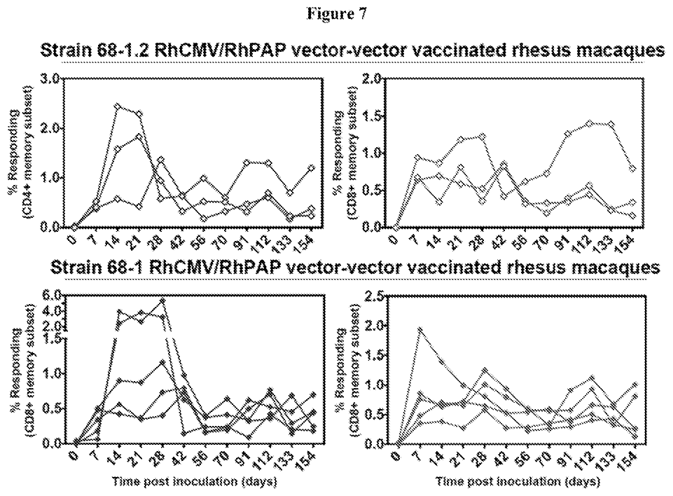

Eight male RM were inoculated with either 68-1.2/PAP, 68-1/PAP, or 68-1/PAPΔRh189 (US11). CD4+ and CD8+ T cell responses in PBMC were measured by ICS using overlapping peptide pools for TNFα and IFNγ at the indicated time points (FIG. 7). The frequency of PAP-specific T cells among memory T cells is shown in FIG. 7, showing that the immunized RM elicited high frequency T cell responses.

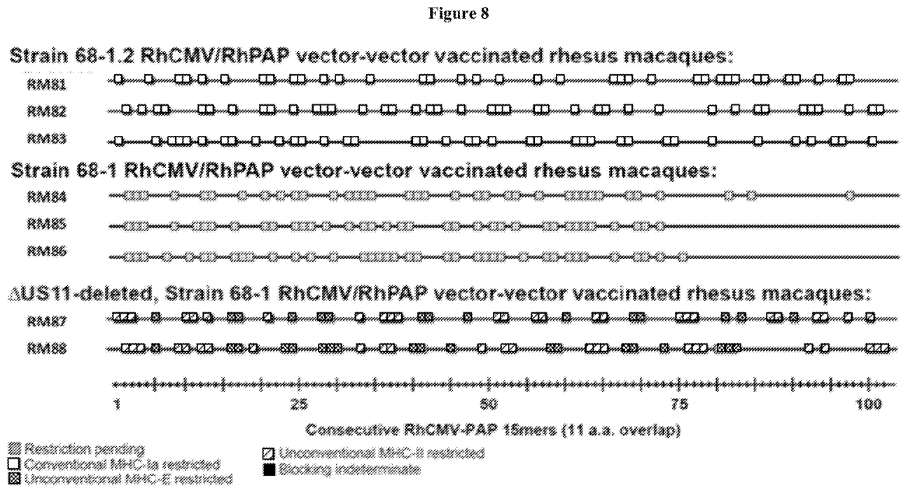

Next, restriction analysis of PAP-specific CD8+ T cells was performed (FIG. 8). CD8+ T cell responses to individual peptides are shown as squares along the PAP sequences. Peptide responses blocked by MHC-I specific antibody W6/32 (but not by peptide VL9) are shown in white boxes. Peptide responses blocked by MHC-II specific peptide CLIP are shown with dashed boxes. Peptide responses blocked by MHC-E-specific peptide are shown with dotted boxes. As expected, CD8+ T cells from 68-1.2/PAP immunized animals were exclusively restricted by MHC-I whereas 68-1/PAP-immunized animals were restricted by MHC-II or MHC-E. Interestingly, deletion of Rh189/US11 did not result in the additional induction of “canonical” MHC-I restricted responses as observed for viral antigens (Hansen, Science 2013). However, this observation is consistent with PAP being subject to “central” immunological tolerance, i.e., CD8+ T cells expressing high affinity TCRs being eliminated by negative selection in the thymus. This result also suggests that the MHC-I restricted CD8+ T cells elicited by 68-1.2/PAP are “non-canonical”, i.e., CD8+ T cells recognizing subdominant epitopes.

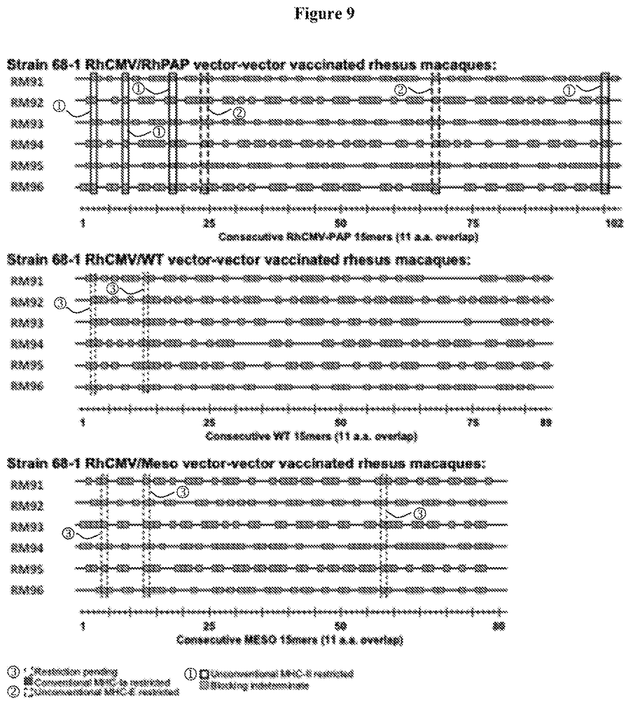

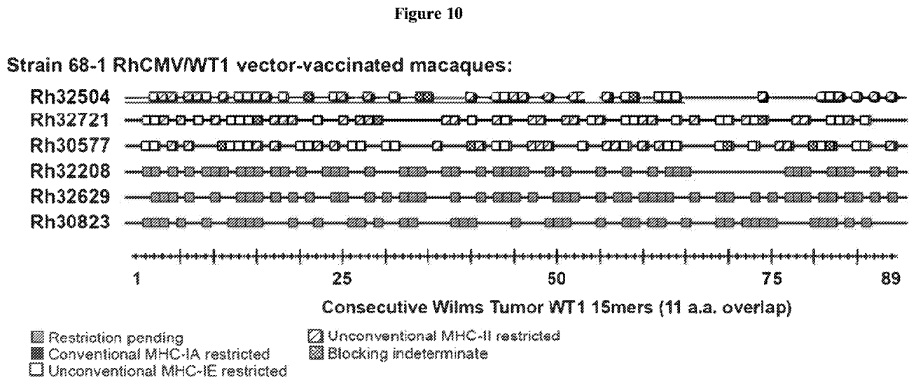

To identify MHC-E and MHC-II-restricted supertopes, restriction analysis was performed for all six animals immunized with 68-1 RhCMV/PAP, 68-1 RhCMV/WT1 (see Example 1), and 68-1 RhCMV/MSNL (see Example 1) (FIG. 9; FIG. 10, shows results for 68-1 RhCMV/WT1 after testing in the presence of blocking reagents). In FIGS. 9 and 10, CD8+ T cell responses to individual peptides are shown as squares along the TAA sequences, and supertope peptides are shown in brackets. The color of the box shows whether the supertopes are MHC-E restricted, MHC-II restricted, or whether the restriction has not yet been determined. The supertope peptides and their sequences are listed in Table 1.

TABLE 1
Supertope Peptides

| | Amino | | SEQ |
|---|---|---|---|
| Antigen | Acid Sequence | Restriction | ID NO |
| PAP #3 | ARAASLSLGFLFLLF | MHC-II | 2 |
| PAP #9 | KELKFVTLVFRHGDR | MHC-II | 3 |
| PAP #18 | QLTQLGMEQHYELGE | MHC-II | 4 |
| PAP #24 | LNESYKHEQVYIRST | MHC-E | 5 |
| PAP #68 | NHMKRATQMPSYKKL | MHC-E | 6 |
| PAP #100 | MVLLFIHIRRGPCWQ | MHC-II | 7 |
| WT1 #3 | VPEPASQHTLRSGPG | MHC-E | 8 |
| WT1 #13 | SAERLQGRRSRGASG | MHC-E | 9 |
| WT1 #14 | LQGRRSRGASGSEPQ | MHC-E | 13 |
| WT1 #58 | HEDPMGQQGSLGEQQ | MHC-E | 14 |
| MSLN #5 | IDESLIFYKKWELEA | Undetermined | 10 |
| MSLN #13 | PFTYEQLDVLKHKLD | Undetermined | 11 |
| MSLN #58 | FMKLRTDAVLPLTVA | Undetermined | 12 |

The MHC-E restricted supertope peptides can be used to identify MHC-E restricted TCRs whereas MHC-II restricted supertope peptides can be used to identify MHC-II restricted TCRs.

What is claimed is:

1. A method of generating an immune response to a tumor-associated antigen in a subject, the method comprising administering to the subject a CMV vector encoding a tumor-associated antigen in an amount effective to elicit a CD8+ T cell response to the tumor-associated antigen, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

2. A method of treating cancer in a subject, the method comprising administering to the subject a CMV vector encoding a tumor-associated antigen in an amount effective to elicit a CD8+ T cell response to the tumor-associated antigen, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

3. A CMV vector encoding a tumor-associated antigen for use in generating an immune response to the tumor-associated antigen in a subject, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

4. A CMV vector encoding a tumor-associated antigen for use in the treatment of cancer in a subject, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

5. Use of a CMV vector encoding a tumor-associated antigen in the manufacture of a medicament for use in generating an immune response to the tumor-associated antigen in a subject, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13), or HEDPMGQQGSLGEQQ (SEQ ID NO: 14).

6. Use of a CMV vector encoding a tumor-associated antigen in the manufacture of a medicament for the treatment of cancer, wherein the CMV vector does not express an active UL128, UL130, UL146, and UL147 protein or orthologs thereof and the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

7. A method of treating cancer caused by a tumor virus in a subject, the method comprising administering to the subject a CMV vector encoding a tumor virus antigen in an amount effective to elicit a CD8+ T cell response to the tumor virus antigen, wherein the CMV vector does not express an active UL128, UL130, UL146, and UL147 protein or orthologs thereof.

8. A CMV vector encoding a tumor virus antigen for use in the treatment of cancer in a subject, wherein the CMV vector does not express an active UL128, UL130, UL146, and UL147 protein or orthologs thereof.

9. Use of a CMV vector encoding a tumor virus antigen in the manufacture of a medicament for the treatment of cancer, wherein the CMV vector does not express an active UL128, UL130, UL146, and UL147 protein or orthologs thereof.

10. The method, CMV vector for use, or use in manufacture of any one of claims 1-6, wherein the tumor-associated antigen comprises the amino acid sequence
(SEQ ID NO: 2)
ARAASLSLGFLFLLF;
(SEQ ID NO: 3)
KELKFVTLVFRHGDR;
(SEQ ID NO: 4)
QLTQLGMEQHYELGE;
(SEQ ID NO: 5)
LNESYKHEQVYIRST;
(SEQ ID NO: 6)
NHMKRATQMPSYKKL;
(SEQ ID NO: 7)
MVLLFIHIRRGPCWQ;
(SEQ ID NO: 8)
VPEPASQHTLRSGPG;
(SEQ ID NO: 9)
SAERLQGRRSRGASG;
(SEQ ID NO: 10)
IDESLIFYKKWELEA;
(SEQ ID NO: 11)
PFTYEQLDVLKHKLD;
(SEQ ID NO: 12)
FMKLRTDAVLPLTVA;
(SEQ ID NO: 13)
LQGRRSRGASGSEPQ;
or
(SEQ ID NO: 14)
HEDPMGQQGSLGEQQ.

11. The method, CMV vector for use, or use in manufacture of any one of claims 1-6, wherein the tumor-associated antigen comprises the amino acid sequence
(SEQ ID NO: 2)
ARAASLSLGFLFLLF.

12. The method, CMV vector for use, or use in manufacture of any one of claims 1-6, wherein the tumor-associated antigen comprises the amino acid sequence
(SEQ ID NO: 3)
KELKFVTLVFRHGDR.

13. The method, CMV vector for use, or use in manufacture of any one of claims 1-6, wherein the tumor-associated antigen comprises the amino acid sequence
(SEQ ID NO: 4)
QLTQLGMEQHYELGE.

14. The method, CMV vector for use, or use in manufacture of any one of claims 1-6, wherein the tumor-associated antigen comprises the amino acid sequence
(SEQ ID NO: 5)
LNESYKHEQVYIRST.

15. The method, CMV vector for use, or use in manufacture of any one of claims 1-6, wherein the tumor-associated antigen comprises the amino acid sequence
(SEQ ID NO: 6)
NHMKRATQMPSYKKL.

16. The method, CMV vector for use, or use in manufacture of any one of claims 1-6, wherein the tumor-associated antigen comprises the amino acid sequence
(SEQ ID NO: 7)
MVLLFIHIRRGPCWQ.

17. The method, CMV vector for use, or use in manufacture of any one of claims 1-6, wherein the tumor-associated antigen comprises the amino acid sequence
(SEQ ID NO: 8)
VPEPASQHTLRSGPG.

18. The method, CMV vector for use, or use in manufacture of any one of claims 1-6, wherein the tumor-associated antigen comprises the amino acid sequence
(SEQ ID NO: 9)
SAERLQGRRSRGASG.

19. The method, CMV vector for use, or use in manufacture of any one of claims 1-6, wherein the tumor-associated antigen comprises the amino acid sequence
(SEQ ID NO: 10)
IDESLIFYKKWELEA.

20. The method, CMV vector for use, or use in manufacture of any one of claims 1-6, wherein the tumor-associated antigen comprises the amino acid sequence
(SEQ ID NO: 11)
PFTYEQLDVLKHKLD.

21. The method, CMV vector for use, or use in manufacture of any one of claims 1-6, wherein the tumor-associated antigen comprises the amino acid sequence
(SEQ ID NO: 12)
FMKLRTDAVLPLTVA.

22. The method, CMV vector for use, or use in manufacture of any one of claims 1-6, wherein the tumor-associated antigen comprises the amino acid sequence
(SEQ ID NO: 13)
LQGRRSRGASGSEPQ.

23. The method, CMV vector for use, or use in manufacture of any one of claims 1-6, wherein the tumor-associated antigen comprises the amino acid sequence
(SEQ ID NO: 14)
HEDPMGQQGSLGEQQ.

24. The method, CMV vector for use, or use in manufacture of any one of claims 1-23, wherein at least 10% of the CD8+ T cells elicited by the CMV vector are restricted by MHC-E or an ortholog thereof, or MHC-II or an ortholog thereof.

25. The method, CMV vector for use, or use in manufacture of claim 24, wherein at least 20%, at least 30%, at least 40%, at least 50%, at least 60% or at least 75% of the CD8+ T cells elicited by the CMV vector are restricted by MHC-E or an ortholog thereof.

26. The method, CMV vector for use, or use in manufacture of any one of claims 1-25, wherein fewer than 10% of the CD8+ T cells elicited by the CMV vector are restricted by MHC-class 1a or an ortholog thereof.

27. The method, CMV vector for use, or use in manufacture of any one of claims 1-26, wherein some of the CD8+ T cells restricted by MHC-E recognize an epitope shared by at least 90% of other subjects immunized with the vector.

28. The method, CMV vector for use, or use in manufacture of any one of claims 1-6 and 10-27, wherein the epitope recognized by the CD8+ T cells comprises a peptide derived from prostatic acidic phosphatase.

29. The method, CMV vector for use, or use in manufacture of any one of claims 1-6 and 10-27, wherein the epitope recognized by the CD8+ T cells comprises a peptide derived from Wilms tumor suppressor protein.

30. The method, CMV vector for use, or use in manufacture of claim 28, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO: 5.

31. The method, CMV vector for use, or use in manufacture of claim 28, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO:6.

32. The method, CMV vector for use, or use in manufacture of claim 29 wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO:8.

33. The method, CMV vector for use, or use in manufacture of claim 29, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO:9.

34. The method, CMV vector for use, or use in manufacture of claim 29, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO:13.

35. The method, CMV vector for use, or use in manufacture of claim 29, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO:14.

36. The method, CMV vector for use, or use in manufacture of claim 24, wherein some of the CD8+ T cells restricted by MHC-II recognize an epitope shared by at least 90% of other subjects immunized with the vector.

37. The method, CMV vector for use, or use in manufacture of claim 36, wherein the epitope comprises a peptide derived from prostatic acidic phosphatase.

38. The method, CMV vector for use, or use in manufacture of claim 37, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO: 2.

39. The method, CMV vector for use, or use in manufacture of claim 37, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO: 3.

40. The method, CMV vector for use, or use in manufacture of claim 37, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO: 4.

41. The method, CMV vector for use, or use in manufacture of claim 37, wherein the epitope recognized by the CD8+ T cells has at least 50%, at least 60%, at least 70%, at least 75%, at least 80%, at least 85%, at least 90%, or at least 95% identity to the amino acid sequence corresponding to SEQ ID NO: 7.

42. A method of generating CD8+ T cells that recognize MHC-E-tumor-associated antigen peptide complexes, the method comprising:
(a) administering to a first subject a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof,
(b) identifying a first CD8+ TCR from the first set of CD8+ T cells, wherein the first CD8+ TCR recognizes a MHC-E/tumor-associated antigen-derived peptide complex;
(c) isolating a second set of one or more CD8+ T cells from a second subject; and
(d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-E/tumor-associated antigen peptide complexes tumor-associated antigen.

43. A method of generating CD8+ T cells that recognize MHC-E-tumor-associated antigen peptide complexes, the method comprising:
(a) isolating from a first subject a first set of CD8+ T cells, wherein the first subject has been administered a recombinant CMV vector comprising a nucleic acid that expresses a tumor-associated antigen, in an amount effective to generate a first set of CD8+ T cells that recognize MHC-E/peptide complexes, wherein the CMV vector does not express an active UL128, UL130, UL146, and UL147 protein or orthologs thereof,
(b) identifying a first CD8+ TCR from the first set of CD8+ T cells, wherein the first CD8+ TCR recognizes a MHC-E/tumor-associated antigen-derived peptide complex;
(c) isolating a second set of one or more CD8+ T cells from a second subject; and
(d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-E/tumor-associated antigen peptide complexes.

44. The method of claim 42-43, wherein the recombinant CMV vector is a recombinant human CMV vector or a recombinant rhesus macaque CMV vector.

45. The method of claim 42-44, wherein the tumor-associated antigen is related to prostate cancer, kidney cancer, mesothelioma, breast cancer, and cervical cancer.

46. The method of any one of claims 42-45, wherein the tumor-associated antigen is prostatic acidic phosphatase, Wilms tumor suppressor protein, mesothelin, and Her-2, or orthologs thereof.

47. The method of claim 46, wherein the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

48. The method, CMV vector for use, or use in manufacture of any one of claim 47, wherein the tumor-associated antigen comprises the amino acid sequence ARAASLSLGFLFLLF (SEQ ID NO: 2).

49. The method, CMV vector for use, or use in manufacture of any one of claim 47, wherein the tumor-associated antigen comprises the amino acid sequence KELKFVTLVFRHGDR (SEQ ID NO: 3).

50. The method, CMV vector for use, or use in manufacture of any one of claim 47, wherein the tumor-associated antigen comprises the amino acid sequence
(SEQ ID NO: 4)
QLTQLGMEQHYELGE.

51. The method, CMV vector for use, or use in manufacture of any one of claim 47, wherein the tumor-associated antigen comprises the amino acid sequence LNESYKHEQVYIRST (SEQ ID NO: 5).

52. The method, CMV vector for use, or use in manufacture of any one of claim 47, wherein the tumor-associated antigen comprises the amino acid sequence
(SEQ ID NO: 6)
NHMKRATQMPSYKKL.

53. The method, CMV vector for use, or use in manufacture of any one of claim 47, wherein the tumor-associated antigen comprises the amino acid sequence MVLLFIHIRRGPCWQ (SEQ ID NO: 7).

54. The method, CMV vector for use, or use in manufacture of any one of claim 47, wherein the tumor-associated antigen comprises the amino acid sequence VPEPASQHTLRSGPG (SEQ ID NO: 8).

55. The method, CMV vector for use, or use in manufacture of any one of claim 47, wherein the tumor-associated antigen comprises the amino acid sequence SAERLQGRRSRGASG (SEQ ID NO: 9).

56. The method, CMV vector for use, or use in manufacture of any one of claim 47, wherein the tumor-associated antigen comprises the amino acid sequence IDESLIFYKKWELEA (SEQ ID NO: 10).

57. The method, CMV vector for use, or use in manufacture of any one of claim 47, wherein the tumor-associated antigen comprises the amino acid sequence PFTYEQLDVLKHKLD (SEQ ID NO: 11).

58. The method, CMV vector for use, or use in manufacture of any one of claim 47, wherein the tumor-associated antigen comprises the amino acid sequence
(SEQ ID NO: 12)
FMKLRTDAVLPLTVA.

59. The method, CMV vector for use, or use in manufacture of any one of claim 47, wherein the tumor-associated antigen comprises the amino acid sequence LQGRRSRGASGSEPQ (SEQ ID NO: 13).

60. The method, CMV vector for use, or use in manufacture of any one of claim 47, wherein the tumor-associated antigen comprises the amino acid sequence
(SEQ ID NO: 14)
HEDPMGQQGSLGEQQ.

61. The method of any one of claims 42-60, wherein the first CD8+ T cell recognizes specific MHC-E supertopes.

62. The method of claim 61, wherein the specific MHC-E supertopes comprise peptides derived from prostatic acidic phosphatase epitopes.

63. The method of claim 61, wherein the specific MHC-E supertopes comprise peptides derived from Wilms tumor suppressor protein epitopes.

64. The method of any one of claims 42-62, wherein the MHC-E supertope has at least 50%, at least 60%, at least 70%, at least 75%, at least 80%, at least 85%, at least 90%, or at least 95% identity to the amino acid sequence corresponding to SEQ ID NO: 5.

65. The method of any one of claims 42-62, wherein the MHC-E supertope has at least 50%, at least 60%, at least 70%, at least 75%, at least 80%, at least 85%, at least 90%, or at least 95% identity to the amino acid sequence corresponding to SEQ ID NO: 6.

66. The method of any one of claims 42-63, wherein the MHC-E supertope has at least 50%, at least 60%, at least 70%, at least 75%, at least 80%, at least 85%, at least 90%, or at least 95% identity to the amino acid sequence corresponding to SEQ ID NO: 8.

67. The method of any one of claims 42-63, wherein the MHC-E supertope has at least 50%, at least 60%, at least 70%, at least 75%, at least 80%, at least 85%, at least 90%, or at least 95% identity to the amino acid sequence corresponding to SEQ ID NO: 9.

68. The method of any one of claims 42-63, wherein the MHC-E supertope has at least 50%, at least 60%, at least 70%, at least 75%, at least 80%, at least 85%, at least 90%, or at least 95% identity to the amino acid sequence corresponding to SEQ ID NO:13.

69. The method of any one of claims 42-63, wherein the MHC-E supertope has at least 50%, at least 60%, at least 70%, at least 75%, at least 80%, at least 85%, at least 90%, or at least 95% identity to the amino acid sequence corresponding to SEQ ID NO:14.

70. The method of any one of claims 42-69, wherein the second CD8+ T cell recognizes specific MHC-E supertopes.

71. The method of claim 70, wherein the specific MHC-E supertopes comprise peptides derived from prostatic acidic phosphatase epitopes.

72. The method of claim 70, wherein the specific MHC-E supertopes comprise peptides derived from Wilms tumor suppressor protein.

73. The method of claim 71, wherein the MHC-E supertope has at least 50%, at least 60%, at least 70%, at least 75%, at least 80%, at least 85%, at least 90%, or at least 95% identity to the amino acid sequence corresponding to SEQ ID NO: 5.

74. The method of claim 71, wherein the MHC-E supertope has at least 50%, at least 60%, at least 70%, at least 75%, at least 80%, at least 85%, at least 90%, or at least 95% identity to the amino acid sequence corresponding to SEQ ID NO: 6.

75. The method of claim 72, wherein the MHC-E supertope has at least 50%, at least 60%, at least 70%, at least 75%, at least 80%, at least 85%, at least 90%, or at least 95% identity to the amino acid sequence corresponding to SEQ ID NO: 8.

76. The method of claim 72, wherein the MHC-E supertope has at least 50%, at least 60%, at least 70%, at least 75%, at least 80%, at least 85%, at least 90%, or at least 95% identity to the amino acid sequence corresponding to SEQ ID NO: 9.

77. The method of claim 72, wherein the MHC-E supertope has at least 50%, at least 60%, at least 70%, at least 75%, at least 80%, at least 85%, at least 90%, or at least 95% identity to the amino acid sequence corresponding to SEQ ID NO:13.

78. The method of claim 72, wherein the MHC-E supertope has at least 50%, at least 60%, at least 70%, at least 75%, at least 80%, at least 85%, at least 90%, or at least 95% identity to the amino acid sequence corresponding to SEQ ID NO:14.

79. The method of any one of claims 42-78, wherein the first CD8+ TCR is identified by DNA or RNA sequencing.

80. The method of any one of claims 42-79, wherein the nucleic acid sequence encoding the second CD8+ TCR is identical to the nucleic acid sequence encoding the first CD8+ TCR.

81. The method of any one of claims 42-80, wherein the first subject and/or the second subject is a human or nonhuman primate.

82. The method of any one of claims 42-81, wherein the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR.

83. The method of any one of claims 42-81 wherein the second CD8+ TCR comprises the non-human primate CDR1α, CDR2α, CDR3α, CDR1β, CDR2β, and CDR3β of the first CD8+ TCR.

84. The method of any one of claims 42-83, wherein the second CD8+ TCR comprises CDR1α, CDR2α, CDR3α, CDR1β, CDR2β, and CDR3β of the first CD8+ TCR.

85. The method of any one of claims 42-84, wherein the nucleic acid sequence encoding the second CD8+ TCR is identical to the nucleic acid sequence encoding the first CD8+ TCR.

86. The method of any one of claims 42-85, wherein the second CD8+ TCR is a chimeric CD8+ TCR.

87. The method of any one of claims 42-86, wherein the second CD8+ TCR comprises CDR1α, CDR2α, CDR3α, CDR1β, CDR2β, and CDR3β of the first CD8+ TCR.

88. The method of any one of claims 42-87, wherein administering the CMV vector to the first subject comprises intravenous, intramuscular, intraperitoneal, or oral administration of the CMV vector to the first subject.

89. The method of any one of claims 42-88, further comprising administering the transfected CD8+ T cells to the second subject to treat or prevent cancer.

90. The method of claim 54, wherein the cancer is prostate cancer, kidney cancer, mesothelioma, breast cancer, and cervical cancer.

91. A method of CD8+ T cells that recognize MHC-II-tumor peptide complexes, the method comprising:
(a) administering to a first subject a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof;
(b) identifying a first CD8+ TCR from the first set of CD8+ T cells, wherein the first CD8+ TCR recognizes a MHC-II/tumor antigen-derived peptide complex;
(c) isolating a second set of one or more CD8+ T cells from a second subject; and
(d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize MHC-II/tumor antigen peptide complexes.

92. A method of generating CD8+ T cells that recognize MHC-II-tumor antigen peptide complexes, the method comprising:
(a) isolating from a first subject a first set of CD8+ T cells, wherein the first subject has been administered a recombinant CMV vector comprising a nucleic acid that expresses a tumor antigen, in an amount effective to generate a first set of CD8+ T cells that recognize MHC-II/peptide complexes, wherein the CMV vector does not express an active UL128, UL130, UL146, and UL147 protein or orthologs thereof,
(b) identifying a first CD8+ TCR from the first set of CD8+ T cells, wherein the first CD8+ TCR recognizes a MHC-II/tumor antigen-derived peptide complex;
(c) isolating a second set of one or more CD8+ T cells from a second subject; and
(d) transfecting the second set of one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating CD8+ T cells that recognize a MHC-II/tumor antigen peptide complexes.

93. The method of claim 91-92, wherein the at least one recombinant CMV vector is a recombinant human CMV vector or a recombinant rhesus macaque CMV vector.

94. The method of claim 91 or 93, wherein the at least one recombinant CMV vector does not express an active UL128 protein, or ortholog thereof, does not express an active UL130 protein, or ortholog thereof, does not express and active UL146, or ortholog thereof, does not express an active UL147, or ortholog thereof, and does not express an active US11 protein, or ortholog thereof.

95. The method of any one of claims 91-94, wherein the mutations in the nucleic acid sequence encoding UL128, UL130, UL146, UL147 or US11 are one or more of point mutations, frameshift mutations, truncation mutations, and deletion of all of the nucleic acid sequence encoding the viral protein.

96. The method of any one of claims 91-95, wherein the tumor-associated antigen is related to prostate cancer, kidney cancer, mesothelioma, breast cancer, and cervical cancer.

97. The method of claim 96, wherein the tumor-associated antigen is prostatic acidic phosphatase, Wilms tumor suppressor protein, mesothelin, and Her-2, or orthologs thereof.

98. The method of claim 97, wherein the tumor-associated antigen comprises the amino acid sequence ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); or HEDPMGQQGSLGEQQ (SEQ ID NO: 14).

99. The method, CMV vector for use, or use in manufacture of any one of claim 98, wherein the tumor-associated antigen comprises the amino acid sequence ARAASLSLGFLFLLF (SEQ ID NO: 2).

100. The method, CMV vector for use, or use in manufacture of any one of claim 98, wherein the tumor-associated antigen comprises the amino acid sequence KELKFVTLVFRHGDR (SEQ ID NO: 3).

101. The method, CMV vector for use, or use in manufacture of any one of claim 98, wherein the tumor-associated antigen comprises the amino acid sequence
(SEQ ID NO: 4)
QLTQLGMEQHYELGE.

102. The method, CMV vector for use, or use in manufacture of any one of claim 98, wherein the tumor-associated antigen comprises the amino acid sequence LNESYKHEQVYIRST (SEQ ID NO: 5).

103. The method, CMV vector for use, or use in manufacture of any one of claim 98, wherein the tumor-associated antigen comprises the amino acid sequence
(SEQ ID NO: 6)
NHMKRATQMPSYKKL.

104. The method, CMV vector for use, or use in manufacture of any one of claim 98, wherein the tumor-associated antigen comprises the amino acid sequence MVLLFIHIRRGPCWQ (SEQ ID NO: 7).

105. The method, CMV vector for use, or use in manufacture of any one of claim 98, wherein the tumor-associated antigen comprises the amino acid sequence VPEPASQHTLRSGPG (SEQ ID NO: 8).

106. The method, CMV vector for use, or use in manufacture of any one of claim 98, wherein the tumor-associated antigen comprises the amino acid sequence SAERLQGRRSRGASG (SEQ ID NO: 9).

107. The method, CMV vector for use, or use in manufacture of any one of claim 98, wherein the tumor-associated antigen comprises the amino acid sequence IDESLIFYKKWELEA (SEQ ID NO: 10).

108. The method, CMV vector for use, or use in manufacture of any one of claim 98, wherein the tumor-associated antigen comprises the amino acid sequence PFTYEQLDVLKHKLD (SEQ ID NO: 11).

109. The method, CMV vector for use, or use in manufacture of any one of claim 98, wherein the tumor-associated antigen comprises the amino acid sequence
(SEQ ID NO: 12)
FMKLRTDAVLPLTVA.

110. The method, CMV vector for use, or use in manufacture of any one of claim 98, wherein the tumor-associated antigen comprises the amino acid sequence LQGRRSRGASGSEPQ (SEQ ID NO: 13).

111. The method, CMV vector for use, or use in manufacture of any one of claim 98, wherein the tumor-associated antigen comprises the amino acid sequence
(SEQ ID NO: 14)
HEDPMGQQGSLGEQQ.

112. The method of any one of claims 91-111, wherein the first CD8+ T cell recognizes a MHC-II supertope.

113. The method of claim 62, wherein the MHC-II supertope comprises a peptide derived from a prostatic acidic phosphatase epitope.

114. The method of any one of claim 113, wherein the MHC-II supertope has at least 50%, at least 60%, at least 70%, at least 75%, at least 80%, at least 85%, at least 90%, or at least 95% identity to the amino acid sequence corresponding to SEQ ID NO: 2.

115. The method of any one of claim 113, wherein the MHC-II supertope has at least 50%, at least 60%, at least 70%, at least 75%, at least 80%, at least 85%, at least 90%, or at least 95% identity to the amino acid sequence corresponding to SEQ ID NO: 3.

116. The method of any one of claim 113, wherein the MHC-II supertope has at least 50%, at least 60%, at least 70%, at least 75%, at least 80%, at least 85%, at least 90%, or at least 95% identity to the amino acid sequence corresponding to SEQ ID NO: 4.

117. The method of any one of claim 113, wherein the MHC-II supertope has at least 50%, at least 60%, at least 70%, at least 75%, at least 80%, at least 85%, at least 90%, or at least 95% identity to the amino acid sequence corresponding to SEQ ID NO: 7.

118. The method of any one of claims 91-117, wherein the second CD8+ T cell recognizes a MHC-II supertope.

119. The method of claim 118, wherein the MHC-II supertope comprises a peptide derived from a prostatic acidic phosphatase epitope.

120. The method of any one of claim 118, wherein the MHC-II supertope has at least 50%, at least 60%, at least 70%, at least 75%, at least 80%, at least 85%, at least 90%, or at least 95% identity to the amino acid sequence corresponding to SEQ ID NO: 2.

121. The method of any one of claim 118, wherein the MHC-II supertope has at least 50%, at least 60%, at least 70%, at least 75%, at least 80%, at least 85%, at least 90%, or at least 95% identity to the amino acid sequence corresponding to SEQ ID NO: 3.

122. The method of any one of claim 118, wherein the MHC-II supertope has at least 50%, at least 60%, at least 70%, at least 75%, at least 80%, at least 85%, at least 90%, or at least 95% identity to the amino acid sequence corresponding to SEQ ID NO: 4.

123. The method of any one of claim 118, wherein the MHC-II supertope has at least 50%, at least 60%, at least 70%, at least 75%, at least 80%, at least 85%, at least 90%, or at least 95% identity to the amino acid sequence corresponding to SEQ ID NO: 7.

124. The method of any one of claims 91-123, wherein the first CD8+ TCR is identified by DNA or RNA sequencing.

125. The method of any one of claims 91-124, wherein the nucleic acid sequence encoding the second CD8+ TCR is identical to the nucleic acid sequence encoding the first CD8+ TCR.

126. The method of any one of claims 91-125, wherein the first subject and/or the second subject is a human or nonhuman primate.

127. The method of any one of claims 91-126, wherein the second subject is a human or nonhuman primate.

128. The method of any one of claims 91-127, wherein the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR.

129. The method of any one of claims 91-127, wherein the second CD8+ TCR comprises the non-human primate CDR1α, CDR2α, CDR3α, CDR1β, CDR2β, and CDR3β of the first CD8+ TCR.

130. The method of any one of claims 91-127, wherein the second CD8+ TCR comprises CDR1α, CDR2α, CDR3α, CDR1β, CDR2β, and CDR3β of the first CD8+ TCR.

131. The method of any one of claims 91-130, wherein the nucleic acid sequence encoding the second CD8+ TCR is identical to the nucleic acid sequence encoding the first CD8+ TCR.

132. The method of any one of claims 91-131, wherein the second CD8+ TCR is a chimeric CD8+ TCR.

133. The method of any one of claims 91-132, wherein the second CD8+ TCR comprises CDR1α, CDR2α, CDR3α, CDR1β, CDR2β, and CDR3β of the first CD8+ TCR.

134. The method of any one of claims 91-133, wherein administering the CMV vector to the first subject comprises intravenous, intramuscular, intraperitoneal, or oral administration of the CMV vector to the first subject.

135. The method of any one of claims 91-134, further comprising administering the transfected CD8+ T cells to the second subject to treat cancer.

136. The method of claim 135, wherein the cancer is prostate cancer, kidney cancer, mesothelioma, breast cancer, and cervical cancer.

137. A CD8+ T cell generated by the method of claims 42-136.

138. A method of treating or preventing cancer in a subject in need thereof, the method comprising administering the CD8+ T cell of claim 137 to the subject.

139. The CD8+ T cell of claim 137 for use in the treatment or prevention of cancer in a subject.

140. Use of the CD8+ T cell of claim 137 in the manufacture of a medicament for the treatment or prevention of cancer.

141. A method of inducing an immune response to a host self-antigen, the method comprising administering the CD8+ T cell claim 137 to the subject.

142. The CD8+ T cell of claim 137 for use in inducing an immune response to a host self-antigen in a subject.

143. Use of the CD8+ T cell of claim 137 in the manufacture of a medicament for inducing an immune response to a host self-antigen.

144. An isolated MHC-E or MHC-II supertope peptide between about 8 and about 15 amino acids in length that is capable of being recognized by CD8+ T cell receptors, wherein the supertope comprises a tumor-associated antigen.

145. The supertope peptide of claim 144, wherein the peptide is selected from the group consisting of: ARAASLSLGFLFLLF (SEQ ID NO: 2); KELKFVTLVFRHGDR (SEQ ID NO: 3); QLTQLGMEQHYELGE (SEQ ID NO: 4); LNESYKHEQVYIRST (SEQ ID NO: 5); NHMKRATQMPSYKKL (SEQ ID NO: 6); MVLLFIHIRRGPCWQ (SEQ ID NO: 7); VPEPASQHTLRSGPG (SEQ ID NO: 8); SAERLQGRRSRGASG (SEQ ID NO: 9); IDESLIFYKKWELEA (SEQ ID NO: 10); PFTYEQLDVLKHKLD (SEQ ID NO: 11); FMKLRTDAVLPLTVA (SEQ ID NO: 12); LQGRRSRGASGSEPQ (SEQ ID NO: 13); and HEDPMGQQGSLGEQQ (SEQ ID NO: 14).

146. A method of overcoming immune tolerance to a tumor-associated antigen in a subject in need thereof, the method comprising administering an effective amount of a cytomegalovirus (CMV) vector that expresses the tumor-associated antigen to the subject.

147. The method of claim 146, wherein the CMV vector is a human CMV vector or a rhesus macaque CMV vector.

148. The method of claim 146, wherein the CMV vector does not express active UL128, or orthologs thereof, does not express active UL130, or orthologs thereof, does not express active UL146, or orthologs thereof, and does not express active UL147, or orthologs thereof.

149. The method of claim 146, wherein the CMV vector does not express active UL128, UL130, UL146, or UL147, or orthologs thereof, due to the presence of one or more mutations in the nucleic acid sequence encoding UL128, UL130, UL146, or UL147.

150. The method of claim 149, wherein the mutations in the nucleic acid sequence encoding UL128, UL130, UL146, or UL147 are one or more of point mutations, frameshift mutations, truncation mutations, and deletion of all of the nucleic acid sequence encoding the viral protein.

151. The method of claims 146-149, wherein the CMV vector is rhesus macaque CMV strain 68-1.

152. The method of any one of claims 146-151, wherein the CMV vector does not express an active UL82 protein, or ortholog thereof.

153. The method of claim 152, wherein the CMV vector does not express an active UL82 protein, or ortholog thereof, due to the presence of one or more mutations in the nucleic acid sequence encoding UL82.

154. The method of claim 153, wherein the mutations in the nucleic acid sequence encoding UL82 are one or more of point mutations, frameshift mutations, truncation mutations, and deletion of all of the nucleic acid sequence encoding UL82.

155. The method of any one of claims 145-154, wherein the tumor-associated antigen is derived from a prostate cancer, kidney cancer, mesothelioma, breast cancer, and cervical cancer.

156. The method of any one of claims 145-155, wherein the tumor-associated antigen is prostatic acidic phosphatase, Wilms tumor suppressor protein, mesothelin, or Her-2.

157. The method of any one of claims 145-156, wherein the effective amount comprises an amount effective to elicit a CD8+ T cell response to the tumor-associated antigen in the subject.

158. The method of claim 156, wherein at least 10%, at least 20%, at least 30%, at least 40%, or at least 50% of the CD8+ T cells are restricted by MHC-I or an ortholog thereof.

159. The method of claim 156, further comprising identifying a CD8+ TCR from the CD8+ T cells elicited by the CMV vector, wherein the CD8+ TCR recognizes a MHC-I/tumor antigen-derived peptide complex.

160. The method of claim 158 or 159, wherein the CD8+ TCR is identified by DNA or RNA sequencing.

161. The method of any one of claims 145-159, wherein the subject is a human.
